(19) United States
(12) Patent Application Publication
(10) Pub. No.: US 2018/0325836 A1
(43) Pub. Date: Nov. 15, 2018

(54) ULTRASONIC TRANSDUCERS SUITABLE FOR ULTRASONIC DRUG DELIVERY VIA A SYSTEM, WHICH IS PORTABLE AND WEARABLE BY THE PATIENT

(71) Applicant: BKR IP Holdco LLC, Broomall, PA (US)

(72) Inventor: Bruce K. REDDING, Jr., Broomalll, PA (US)

(21) Appl. No.: 15/774,235

(22) PCT Filed: Nov. 7, 2016
(86) PCT No.: PCT/US2016/060859

§ 371 Date: May 7, 2018

Related U.S. Application Data
(60) Provisional application No. 62/285,668, filed on Nov. 6, 2015.

(51) Int. Cl. A61K 9/70; A61K 9/00; A61M 37/00

(52) U.S. Cl. CPC A61K 9/7023; A61K 38/28; A61M 37/0092; A61K 9/0009

(57) ABSTRACT

The invention is a device for transmitting acoustical energy between an ultrasonic generator, through a medicine-containing patch, and into the exterior surface of the skin of a patient, typically known as an ultrasonic transducer. The ultrasonic transducer emits a sonic transmission of variable intensity and frequency for the purpose of enhancing the absorption through the patient's skin of pharmaceutical compounds stored within a transdermal patch or other medication holding device. The transducers are designed to accommodate an ultrasonic drug delivery system, which is worn by the patient.

### PRIORITY CLAIM, CROSS REFERENCE TO RELATED APPLICATIONS AND INCORPORATION BY REFERENCE

This application is related to, claims priority under, and claims the benefit of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790; “METHOD AND APPARATUS FOR MEASURING THE DOSE REMAINING UPON A TRANSDERMAL DRUG DELIVERY DEVICE”, Bruce K. Redding, Jr., filed on Aug. 1, 2014, and having Ser. No. 61/999,589; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837, PCT applications filed in the United States Patent and Trademark Office: “MODIFIED TRANSDERMAL DELIVERY PATCH WITH MULTIPLE ABSORBENT PADS”, Bruce K. Redding, Jr., filed on Jul. 6, 2015, and having serial number PCT/US/39236; MODIFIED TRANSDERMAL DELIVERY DEVICE OR PATCH AND METHOD OF DELIVERING INSULIN FROM SAID MODIFIED TRANSDERMAL DELIVERY DEVICE, Bruce K. Redding, Jr., filed on Jul. 6, 2015, and having serial number PCT/US15/39264; METHOD FOR GLUCOSE CONTROL IN DIABETICS Bruce K. Redding, Jr., filed on Jul. 6, 2015, PCT/US15/39268.

This application hereby incorporates herein by reference the subject matter disclosed in the written abstracts, descriptions, the drawings and claims, in their entireties of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790; “METHOD AND APPARATUS FOR MEASURING THE DOSE REMAINING UPON A TRANSDERMAL DRUG DELIVERY DEVICE”, Bruce K. Redding, Jr., filed on Aug. 1, 2014, and having Ser. No. 61/999,589; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837; PCT applications filed in the United States Patent and Trademark Office: “MODIFIED TRANSDERMAL DELIVERY PATCH WITH MULTIPLE ABSORBENT PADS”, Bruce K. Redding, Jr., filed on Jul. 6, 2015, and having serial number PCT/US/39236; MODIFIED TRANSDERMAL DELIVERY DEVICE OR PATCH AND METHOD OF DELIVERING INSULIN FROM SAID MODIFIED TRANSDERMAL DELIVERY DEVICE, Bruce K. Redding, Jr., filed on Jul. 6, 2015, and having serial number PCT/US15/39264; METHOD FOR GLUCOSE CONTROL IN DIABETICS Bruce K. Redding, Jr., filed on Jul. 6, 2015, PCT/US15/39268.

### FIELD OF THE INVENTION

The present invention relates to a portable delivery system for administering a medicament to a patient by inducing the transfer of the medicament through the skin. In particular, acoustical energy delivered by a portable, self-powered, programmable ultrasonic transducer placed over a medicament containing patch causes the medicament to be transferred across the skin barrier.

### BACKGROUND OF THE INVENTION

The present invention relates to a portable programmable ultrasonic device, which is worn by the patient, over a transdermal drug delivery patch for the purpose of enhancing the penetration of medicinal compounds (drugs) contained within the transdermal patch, through the skin into the patient's blood stream. Further, the portable ultrasonic applicator may be programmed to apply acoustical energy at different times and thereby cause the delivery of a varying quantity of the medicinal compound over time. The portable ultrasonic applicator may be programmed to deliver a medicinal compound to the patient continuously (sustained release) or in intermittently (pulsed release) whichever may be deemed more appropriate to a drug maintenance and treatment regimen for a particular patient.

In the prior art, transdermal drug delivery systems employ a medicated device or patch, which is affixed to the exposed surface of the skin of a patient. The patch allows a medicinal compound contained within the patch to be absorbed into the skin layers and finally into the patient's blood stream. Transdermal drug delivery avoids the need and the pain associated with drug injections and intravenous drug administration. Transdermal drug delivery also avoids gastrointestinal metabolism of administered drugs, reduces the elimination of drugs by the liver, and provides a sustained release of the administrated drug. Transdermal drug delivery also enhances patient compliance with a drug regimen because of the relative ease of administration and the sustained release of the drug.

Several medicinal compounds are not suitable for transdermal drug delivery since they are absorbed with difficulty through the skin due to the molecular size of the drug or to other bioadhesion properties of the drug. In these cases, when transdermal drug delivery is attempted, the drug may be found pooling merely on the outer surface of the skin and not permeating directly through into the blood stream. Once such example is insulin, which in the prior art has been found difficult to administer by means of transdermal drug delivery.

Some of the most critically needed medications are presently administered either by injection or oral dosage forms. In particular, chemotherapeutic agents are administered in increased dosages because of their need to survive degradation in the gastrointestinal tract. Many critical treatments for AIDS require a cocktail of drugs taken orally in solid dosage forms, several times a day to be effective. These medications are not suitable for transdermal drug delivery use because of the extensive dosing requirement, the inability of the drug molecule to remain stable in a transdermal form. Moreover, the unsuitability for transdermal to skin transfer of the drug leading to low bioabsorbance of the drug across the skin layers.

Generally, conventional transdermal drug delivery methods have been found suitable only for low molecular weight medications such as nitroglycerin for alleviating angina, nicotine for smoking cessation regimens, and estradiol for estrogen replacement in post-menopausal women. Larger molecular medications such as insulin (a polypeptide for the treatment of diabetes), erythropoietin (used to treat severe anemia) and gamma-interferon (used to boost the immune systems cancer fighting ability) are all compounds not normally effective when used with transdermal drug delivery methods of the prior art.

Recent work conducted upon cadavers using ultrasound (Langer, Edwards, Kost-MIT-1995) suggests that ultrasound applied to transdermal delivery devices and patches can enhance the penetration and/or absorption of certain low molecular weight pharmaceutical medications through the skin layer where normally low skin penetration would be expected without the use of the ultrasonic device. It has been shown that sonophoresis, the use of ultrasonic energy to enhance bioabsorption of large protein molecules or large molecule medications through the skin's outer layer, is possible when low frequencies are applied to a transdermal patch for particular medications. U.S. Pat. No. 5,947,921 to Kost et al. describes a clinical apparatus for inducing enhanced drug delivery via ultrasonic treatment.

While this patent describes a method for the use of low frequency ultrasound for particular drug delivery to enhance transdermal drug delivery, the method requires the use in a clinical ultrasonic delivery setting. Moreover the time for delivery for measurable amounts using these methods range from 24 hours to 10 minutes. In this method, the use of ultrasound-transdermal drug delivery treatment is actually less desirable from a patient administration standpoint than a simple injection. This method is undesirable because of the need for the patient to visit the clinical setting and to remain on a treatment table while the ultrasound treatment is used to deliver the drug. This method causes damage to skin because the same area of the skin is treated continuously.

Ultrasound has also been used to enhance permeability of the skin and synthetic membranes to drugs and other molecules. Ultrasound has been defined as mechanical pressure waves with frequencies above 20 kHz, H. Lutz et al., and Manual of Ultrasound 3-12 (1984). Ultrasound is generated by vibrating a piezoelectric crystal or other electromechanical element by passing an alternating current through the material, R. Brucks et al., 6 Pharm. Res. 697 (1989). The use of ultrasound to increase the permeability of the skin to drug molecules has been termed sonophoresis or phonophoresis.

U.S. Pat. No. 4,309,989 to Fahim describes topical application of medications in a coupling agent for the treatment of Herpes virus infections and demidox mite infestations. The medications are massaged into the affected area by ultrasound to cause the medication to penetrate the skin. U.S. Pat. No. 4,372,296 to Fahim similarly describes topical application of zinc sulfate and ascorbic acid in a coupling agent for treatment of acne.

U.S. Pat. No. 4,767,402 to Kost et al. discloses a method for enhancing and controlling infusion of molecules having a low rate of permeability through skin using ultrasound in the frequency range of between 20 kHz and 10 MHz, and in the intensity range of between 0 and 3 W/cm.sub.2. The molecules are either incorporated in a coupling agent or, alternatively, applied through a transdermal patch. Kost et al. further teach that the parameters of time, frequency, and power can be optimized to suit individual situations and differences in permeability of various molecules and of various skins. Transbuccal drug delivery with ultrasound has also been disclosed, U.S. Pat. No. 4,948,587 to Kost et al.

U.S. Pat. No. 5,115,805 to Bommannan et al. discloses the use of specific frequencies (i.e. >10 MHz) of ultrasound to enhance the rate of permeation of drugs through human skin into the body. Frequencies above 10 MHz gave improved penetration of the skin above that described earlier. It is alleged that chemical penetration enhancers and/or iontophoresis can also be used in connection with the ultrasound treatment to enhance delivery of drugs through the skin into the body.

U.S. Pat. No. 5,016,615 to Driller et al. involves local application of a medication by implanting a drug-containing receptacle adjacent to a body tissue to be treated and then applying ultrasound to drive the drug out of the receptacle and into the body tissue. This method has the disadvantage of requiring surgical implantation of the drug receptacle and a noninvasive technique is preferred. U.S. Pat. No. 4,821,740 to Tachibana et al. discloses a kit for providing external medicines that includes a drug-containing layer and an ultrasonic oscillator for releasing the drugs for uptake through the surface of the skin. In U.S. Pat. No. 5,007,438 to Tachibana et al. is described an application kit in which a layer of medication and an ultrasound transducer are disposed within an enclosure. The transducer may be battery powered. Ultrasound causes the medication to move from the device to the skin and then the ultrasound energy can be varied to control the rate of administration through the skin.

Other references teaching the use of ultrasound to deliver drugs through the skin include D. Bommannan et al., 9 Pharmaceutical Res. 559 (1992); D. Bommannan et al., 9 Pharmaceutical Res. 1043 (1992); K. Tachibana, 9 Pharmaceutical Res. 952 (1992); P. Tyle & P. Agrawala, 6 Pharmaceutical Res. 355 (1989); H. Benson et al., 8 Pharmaceutical Res. 1991); D. Levy et al., 83 J. Clin. Invest. 2074 (1989). Other methods of increasing the permeability of skin to drugs have been described, such as ultrasound or iontophoresis. Iontophoresis involves the application of an external electric field and topical delivery of an ionized form of drug or a unionized drug carried with the water flux associated with ion transport (electro-osmosis). While permeation enhancement with iontophoresis has been effective, control of drug delivery and irreversible skin damage are problems associated with the technique.

Thus, while the use of ultrasound for drug delivery is known, results have been largely disappointing in that enhancement of permeability has been relatively low. There is no consensus on the efficacy of ultrasound for increasing drug flux across the skin. While some studies report the success of sonophoresis, J. Davick et al., 68 Phys. Ther. 1672 (1988); J. Griffin et al., 47 Phys. Ther. 594 (1967); J. Griffin & J. Touchstone, 42 Am. J. Phys. Med. 77 (1963); J. Griffin et al., 44 Am. J. Phys. Med. 20 (1965); D. Levy et al., 83 J. Clin. Invest. 2074); D. Bommannan et al., 9 Pharm. Res. 559 (1992), others have obtained negative results, H. Benson et al., 69 Phys. Ther. 113 (1988); J. McElnay et al., 20 Br. J, Clin. Pharmacol. 4221 (1985); H. Pratzel et al., 13 J. Rheumatol. 1122 (1986). Systems in which rodent skin were employed showed the most promising results, whereas systems in which human skin was employed have generally shown disappointing results. It is well known to those skilled in the art that rodent skin is much more permeable than human skin, and consequently the above results do not teach one skilled in the art how to effectively utilize sonophoresis as applied to transdermal delivery and/or monitoring through human skin.

In the above mentioned examples the specific design of the ultrasonic transmitting device, commonly known as a transducer, is not exposed. In fact most references envisioned a typical ultrasonic wand or sonicator for their device, not taking into account the power utilization of the transducer and the size of the device. Applicant envision a device, which is worn by the patient, said device being programmed to deliver an ultrasonic signal through a transdermal patch according to a timing circuit. Accordingly the transducers need to be small and compact. Additionally the transducers need to be powered by a battery, which is also portable and worn by the patient.

Applicant suggest that the parameters of ultrasound that can be changed to improve or control penetration include frequency, intensity, and time of exposure. All three of these parameters may be modulated simultaneously in a complex fashion to increase the effect or efficiency of the ultrasound as it relates to enhancing the transdermal molecular flux rate either into or out of the human body.

Applicant show a method of multiple array transducers that changes the area of the skin used for drug absorption.

Since ultrasound is rapidly attenuated in air, a coupling agent, preferably one having lowest realizable absorption coefficient that is non-staining, non-irritating, and slow drying, may be needed to efficiently transfer the ultrasonic energy from the ultrasound transducer into the skin. When a chemical enhancer fluid or anti-irritant or both are employed, they may function as the coupling agent. For example, glycerin used as an anti-irritant may also function as a coupling agent. If needed, additional components may be added to the enhancer fluid to increase the efficiency of ultrasonic transduction.

In general, ultrasound exposure times for permeation through human skin have been 24 hours to 10 minutes. The exposure may be either continuous or pulsed to reduce heating of biological membranes. Average intensities have been in the range of 0.01-5 W/cm.sup.2 and are selected to be high enough to achieve the desired result and low enough to avoid significant elevation of skin temperature. Frequencies have varied from 20 kHz to 50 MHz, preferably 5-30 MHz. The depth of penetration of ultrasonic energy into living soft tissue is inversely proportional to the frequency, thus high frequencies have been suggested to improve drug penetration through the skin by concentrating the effect in the outermost skin layer, the stratum corneum. Applicant theorize that pharmaceutical agents under sonic transdermal delivery will require variable frequencies and intensities in order to deliver therapeutic quantities of drugs to patients. Applicant further theorize that variables such as fat content and mass of a particular patient's tissue, through which the drug will be delivered, will vary the frequency and intensity requirements to obtain an effective dosing regimen.

Applicant also theorize that the encapsulation of various compounds would increase their permeability and allow slow time release of medication. Applicant further theorize that some excipients can improve transport through the stratum corneum and absorption into the blood stream. Applicant suggest that several drugs can be applied using this method for local application of medication.

Although it has been acknowledged that enhancing permeability of the skin should theoretically make it possible to transport molecules into the body for therapeutic purposes, portable programmable devices and methods have not been disclosed. Because of the inefficiencies and lack of safety of the previous ultrasonic methods, no useful device has been proposed for the transdermal delivery of drugs with an ultrasonic assist.

In view of the foregoing problems and/or deficiencies, the development of a transducer device for safely enhancing the permeability of the skin for noninvasive drug delivery in a more rapid time frame would be a significant advancement in the art.

While these experiments have shown that transdermal drug delivery can be made effective or enhanced for certain larger molecule peptides, proteins and other medications through the use of an ultrasonic assist, the treatments do not provide for patient mobility. Patient mobility, coupled with sustained release of a broad range of drugs, remains the elusive goal of transdermal delivery devices. Systems employing drug delivery via a transdermal patch and a clinical or stationary ultrasonic system, such as demonstrated in U.S. Pat. No. 4,767,402 to Kost et al., are not desirable, not effective, nor functional tools for administering medications to patients through a sustained release format especially over the course of a day or even longer.

### SUMMARY OF THE PRESENT INVENTION

Accordingly, the purpose of this invention is to provide a transducer device for enhancing transdermal drug delivery by the use of ultrasound, especially of larger pharmaceutically active compounds, wherein the transducer device is small in size, battery powered, highly efficient and able to generate an ultrasonic transmission suitable for effecting the transmission of a pharmaceutical compound from a transdermal patch. The present invention is an ultrasonic transducer device, which is placed directly in contact with a transdermal delivery device or patch for the purpose of both enhancing, and controlling the delivery of medications contained within the patch into and through the skin layer of a target patient. The transducer device may be placed directly within a drug-containing patch or worn over the patch, and held in place by adhesive strips or body affixing straps. The transdermal patch may contain a particular medication or cocktail of medications for treatment of disease or relief of pain

Accordingly the primary object of the invention is a transducer device suitable for applying ultrasound to a transdermal patch for controlling transdermal and/or transmucosal flux rates of drugs and other molecules into the body and the bloodstream.

Another object of the invention is a Class V flextensional cymbal transducer and transducer array for use in the device to deliver low frequency ultrasound in a portable device at high efficiency for transdermal drug delivery and therapeutic applications.

A further object of the invention is the improvement of transport of drug molecules through the hair follicles, skin pores and stratum corneum.

Another object of the invention is a method for non-invasive delivery of biologically active molecules through the skin and mucosal membranes using ultrasound.

A further object of this invention is the use of several areas of the skin simultaneously or sequentially by use of multiple transducers and transducer arrays.

These and other objects of the inventions can be accomplished by applying various ultrasound frequencies, intensities, amplitudes and/or phase modulations to control the magnitude of the transdermal flux to achieve a therapeutic or nutritional level.

One aspect of the programmability and flux control is the ability to optimize therapeutic delivery for an individual patient (such examples may include patients that are at different stages of the disease, elderly patients, young, juvenile, or according to gender).

Another aspect is to optimize delivery for each drug. The molecular structure of each biologically active molecule is different and responds differently to ultrasound. Control of the frequency, intensity, concentration, timing of delivery, drug regimen can optimize delivery of each drug type.

A further aspect of the invention is the transducer or array of transducers built into the patch.

A further aspect of the invention is the sliding in of the transducers into the patch.

A preferred aspect of the invention is the use of the transducer device for insulin delivery.

Another embodiment of the invention is the use of phase modulation, alternating waveforms and frequency modulation to achieve more effective ultrasonic transdermal drug transport and to increase the rate of delivery of drugs

Another aspect of the invention is the combination of ultrasound with iontophoresis, electroporation, depilatories, or use with chemical enhancers such as surfactants to facilitate transdermal permeation.

Additional objects, advantages and novel features of the invention will be set forth in part in the description which follows, and in part will become apparent to those skilled in the art upon examination of the following, or may be learned by practice of the invention.

### BRIEF DESCRIPTION OF THE DRAWINGS

FIG. 1 is an artist's depiction of an ultrasonic drug delivery apparatus, which is worn by the patient, as it is placed upon the arm of a patient.

FIG. 2 is an artist's depiction of an alternative ultrasonic drug delivery apparatus, which is worn by the patient, as it is placed upon the abdomen of a patient.

FIG. 3 is an illustration of the structure of human skin.

FIG. 4A illustrates a cross section view of the preferred embodiment of the transducer element of this invention, said transducer element being a “cymbal” type transducer design.

FIG. 4B illustrates the fabrication steps to produce a “cymbal” type transducer element.

FIG. 4C illustrates a cross section view of transducer element a stacked “cymbal” type transducer design designed to provide higher ultrasonic efficiency, intensity and power output.

FIG. 5A is illustrates the dimensions obtained in the preferred embodiment of the transducer design, and the use of a polymer potting used as a resonance compatible coupling agent coating over the surface of the transducer element.

FIG. 5B illustrates the small dimensions obtained in the fabrication of a “cymbal” type transducer element.

FIG. 6. Illustrates an array of transducers used to enhance sonic efficiency and to provide multiple delivery sites to the skin.

FIG. 7. Depicts the use of an alternating waveform, a conversion from sawtooth to square wave, as generated by the frequency driver of this invention.

FIG. 8A shows a scan using a HPLC of insulin, Humilin Regular, supplied by Eli Lilly Co., where no ultrasound was applied.

FIG. 8B shows a scan using a HPLC of insulin, Humilin Regular, supplied by Eli Lilly Co., after the sample was treated with low frequency and low intensity ultrasound continuously over a eight hour period.

FIG. 9 shows an animal study conducted with subject rats, where the transducer array was used in a test for the transdermal delivery of insulin.

FIG. 10 shows the results of glucose analysis in the blood of subject rats, where the transducer array was used in a test for the transdermal delivery of insulin.

FIG. 11 shows that the test area under the ultrasonic transdermal insulin delivery conducted with subject rats, showed no skin damage, where the transducer array was used in a test for the transdermal delivery of insulin.

FIGS. 12 through 23 illustrates the data achieved from the testing of the single cymbal transducer element of this invention.

FIGS. 24 through 28 illustrate the data achieved from the testing of the multi-element transducer array consisting of nine cymbal transducer elements connected in parallel configuration as illustrated in FIG. 6 of this invention.

FIG. 29 illustrate the data achieved from the testing of the multi-element transducer array consisting of nine cymbal transducer elements connected in parallel configuration as illustrated in FIG. 6 of this invention, with regards to power utilization and battery needs.

### DESCRIPTION OF THE PREFERRED EMBODIMENTS

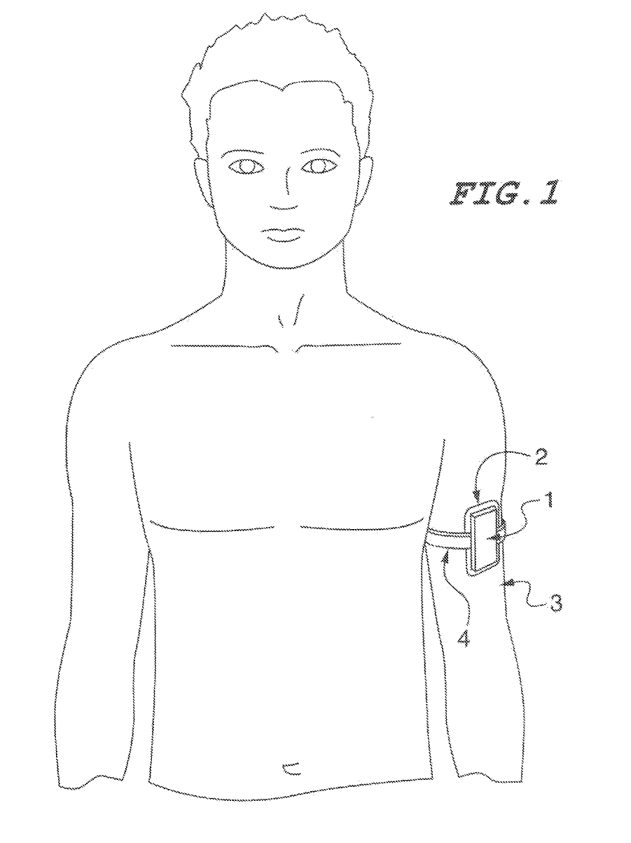

FIG. 1 illustrates a wearable, non-invasive, ultrasonic-transdermal drug delivery system comprising an ultrasonic applicator 1, which is shown placed directly over a transdermal delivery device or patch 2. The applicator and patch are attached to the exterior of the patient's skin 3 by means of a strap 4, which holds the ultrasonic applicator 1 and patch 2 in place. Power for the ultrasonic applicator 1 is provided by power cells (not shown), which are ideally rechargeable, and may be located within the strap 4 itself. Alternatively the power supply may be contained within the ultrasonic applicator device 1 itself or provided by an external source.

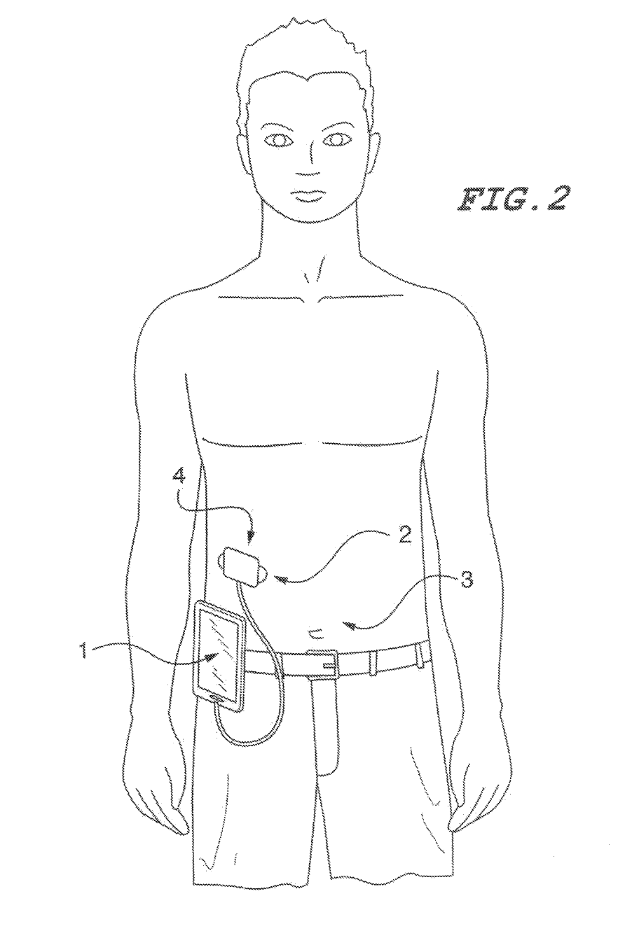

FIG. 1 illustrates that the use is on the arm of the patient. But the system could be placed over the patient's chest (as in the case of nitroglycerin drug delivery) abdomen, (as seen in FIG. 2) or in some other part of the patient's body as determined by the medical personnel administrating the drug treatment regimen. Other body placements are possible including the neck, back and legs.

FIG. 2 shows another embodiment with the ultrasonic applicator 1 affixed directly over the transdermal patch 2 and held onto the bare skin 3, wherein the transducers 4 are placed directly in contact with a transdermal patch 2, in this instance affixed to the patient's abdomen 3.

### Structure of Human Skin and Drug Transport Dynamics.

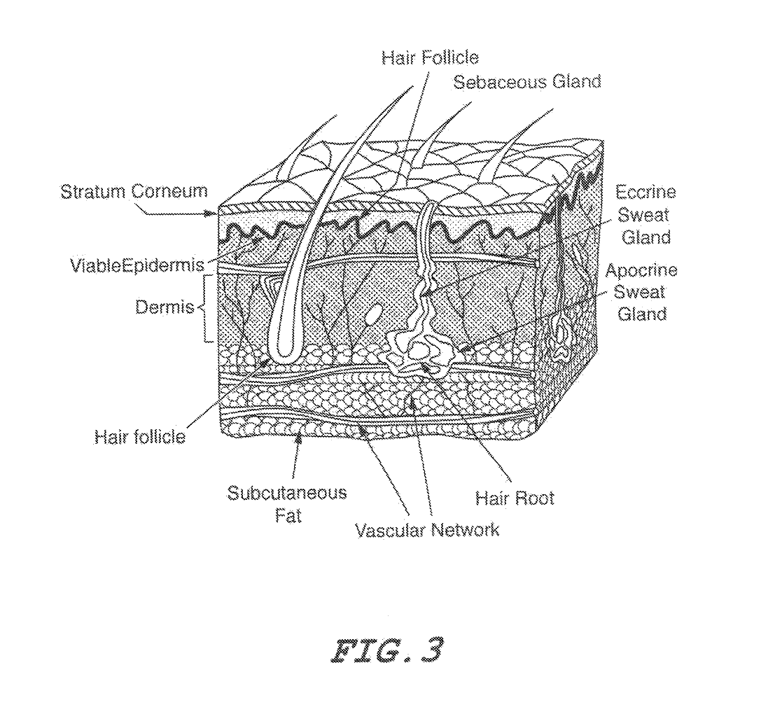

FIG. 3 illustrates the structure of human skin. Essentially there are three pathways through the skin into the bloodstream:
1. Breaching the Stratum Corneum.
2. Passing pharmaceutical agent through pores in the skin.
3. Passing a pharmaceutical agent through the skin by following the hair follicle to the hair root, and from there into the vascular network located at the base of the hair root.

This invention seeks to provide transdermal drug delivery by utilizing drug pathways associated with the pore and the hair follicle system on the patient's skin. Specifically the ultrasonic frequency, intensity level and waveform dynamics are adjusted to maximize drug delivery through the hair follicle pathway primarily and through the pores in the skins surface secondarily, but not directly through the stratum corneum. Applicant have determined that the amount of energy needed for piercing the stratum corneum is excessive and is also damaging to the fatty tissue.

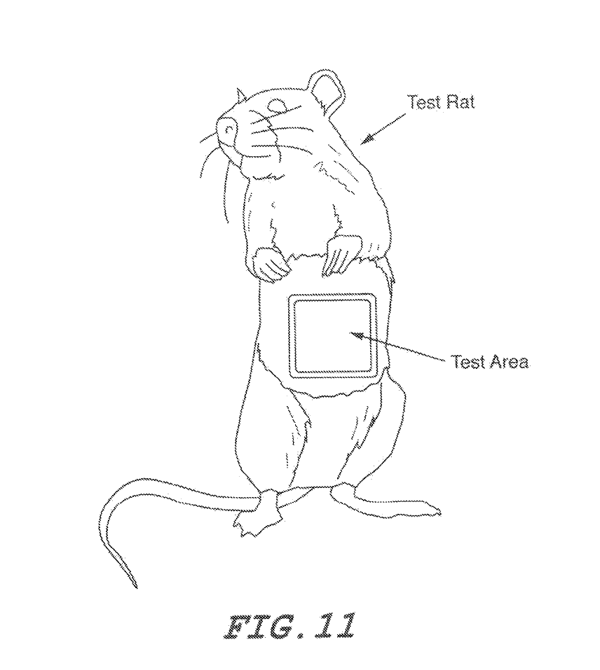

Applicant have also discovered that through the use of alternating waveforms the amount of energy transmitted to the surface of the skin could be minimized while also providing a pressure wave effect upon the skin, enhancing drug delivery through the hair follicle and pore system. Referring to FIG. 11 the preferred embodiment employs a waveform, which alternates from sawtooth to square wave. The amplitude of and intensity of the wave shaping is theorized to aid in both the homogenization of the drug contained within the transdermal patch, helping to miniaturize the beadlet size of the active pharmaceutical substance within the patch, and in drug transport through the skin. Applicant theorize that the short, peaked portion of the ultrasonic waveform in a sawtooth shape helps with drug homogenization, without imparting destructive frequencies and cavitation to the drug substance. Upon conversion to the square waveform the ultrasonic transmission acts to massage and open the fatty tissue surrounding the hair follicle and pores. Drugs permeating from the transdermal patch are in monomer form and/or reduced in droplet size, below 50 Angstroms, making them more suitable in dimension to pass through the skin. The square waveform helps to “push” the drug through the pores and alongside the hair follicles, where the drug makes it way to the hair root, and directly into the bloodstream at the vascular network.

To achieve ultrasound promoted transdermal drug delivery of drugs, the Transdermal Patch should be designed to work in conjunction with the sonic applicator. In particular, the contact between the applicator and the patch must insure efficient acoustic energy transmission. The selection of the materials and adhesives is important to maintain the intensity and power output of the ultrasonic transmission from the transducers through the transdermal patch. Applicant have noted that insulin, one of many active pharmaceutical substances targeted for enhanced drug delivery via this invention, has a large molecule size, and forms hexamers generally over 50 Angstroms, making it difficult to permeate through the pores of the skin. Insulin molecules tend to agglomerate when stored and as a result zinc. Insulin therefore, stored within the patch may tend to agglomerate into even larger drug clump sizes, reducing skin transport potential.

To help alleviate this problem and to keep the drug at a size sufficiently small enough for skin transport the waveform of the ultrasonic signal is altered from time to time, from a sawtooth to a square waveform. FIG. 7 illustrates the alternating waveform concept wherein a sawtooth waveform was found to be more efficient at drug homogenization within the patch, leading to increased skin transport as the ultrasonic waveform switches to a square wave shape. Under the sawtooth waveform the short period leads to high energy, with short duration of pressure amplitude, leading to a vibration effect with the targeted pharmaceutical substance. This vibration is with low heat potential and has the effect of mixing or homogenizing the drug within the patch. Smaller beadlet sizes are made possible by the sawtooth waveform.

In the prior art the drug delivery pathway through the stratum corneum enabled initial quantities of a drug to become permeated through the skin, but as longer periods of ultrasound were applied to the same location on the skin the delivery rate dropped off or was reduced to zero. This implies that ultrasound applied to same site at the skin's surface should not be continued for lengthy periods of time. Applicant theorize that the attempts by the previous art to breach the stratum corneum failed over time because the cavitation eventually over-heated the fatty tissue contained within the epidermis and this effect may have changed the density of the fatty composites within this skin layer. An increase in such density would retard further drug permeation through the skin.

### Design of Transducer Element

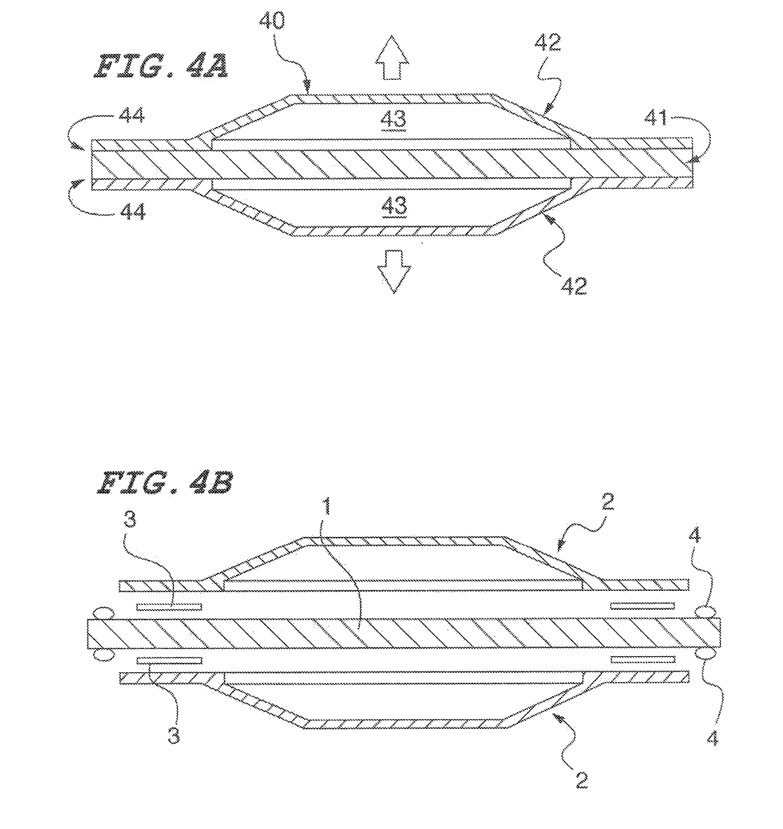

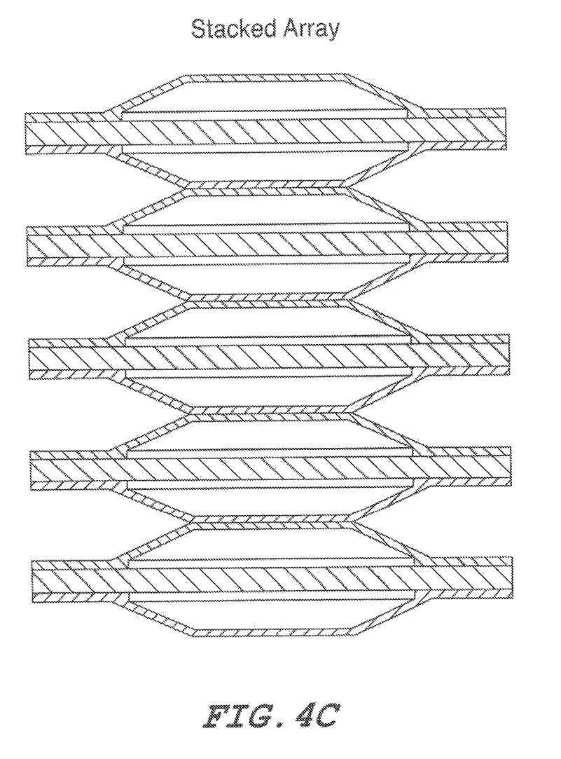

FIG. 4A illustrates the design of a cymbal type of ultrasonic transducer 40, which is the preferred embodiment of the transducer element of this invention. From FIG. 4A it can be seen that a cymbal transducer 40 is based upon a piezoelectric disc 41 such as PZT4 (Piezokinetics Corp. Bellefonte, Pa.), connected to two metal caps 42 composed of titanium foil preferably. FIG. 4A illustrates that there is a hollow air space 43 between the piezoelectric disc 41 and the end caps 42. The end caps 42 are connected to the piezoelectric disc 41 by a non-electrically conductive adhesive 44 to form a bonded layered construction to the transducer 40. The cymbal transducer offers a thin, compact structure more suited for a portable ultrasonic drug delivery apparatus. Additionally this transducer offers greater efficiency for the conversion of electric power to acoustically radiated power. Applicant chose this design of a transducer also because of its potential to be battery powered and its small, lightweight features.

FIG. 4C illustrates the design of a stacked cymbal type of ultrasonic transducer 40, which is the embodiment of the transducer element of this invention. In a stacked transducer construction greater intensity of ultrasonic signals can be achieved. U.S. Pat. No. 5,729,077, Newnham et al, discloses the use of stacked transducers, essentially transducers fitted atop each other, to increase ultrasonic intensities while maintaining a given frequency level. Used in this invention the stacked transducer construction is intended to increase intensity while improving the power utilization of the transducer system.

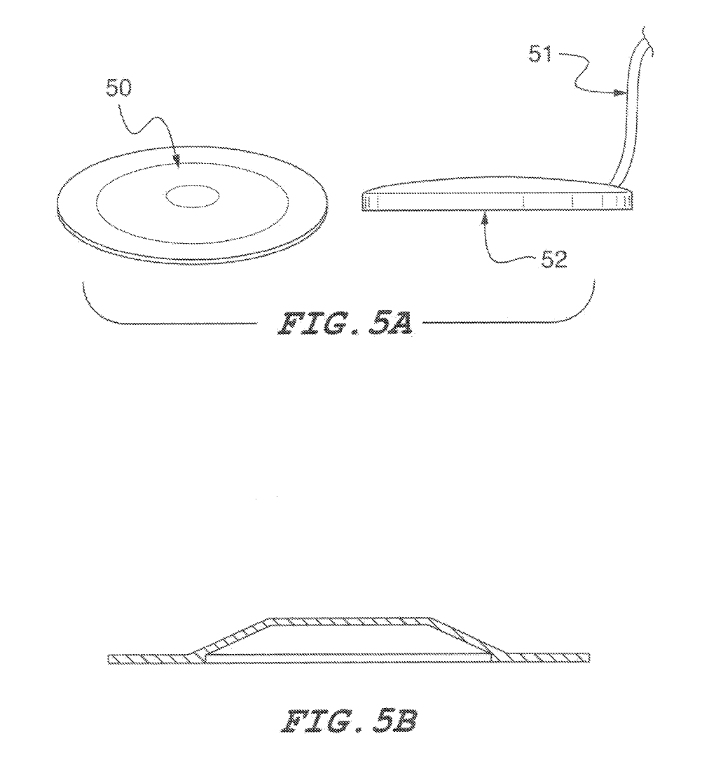

FIG. 5 illustrates that the cymbal transducer enables a compact and minute size to the transducer element of the invention. The sizing of the transducers was obtained at just 0.5″ inches diameter. The small size transducer was necessary in the invention to enable the transducers to fit within the dimensions of the Transdermal Patch. In addition the small size enabled a lower weight potential for the transducers, again aiding in the portability of the invention. The transducer element 50 is a cymbal type construction attached to a power cable 51. The transducer element 50 is coated in a polymer housing 52, ideally composed of uralite resin, which is used to avoid short circuits between the two metallic caps 42 (FIG. 4) and provides acoustic coupling for the sonic transmission.

The cymbal type transducer design offers several key advantages over the prior art:
Compact structure, with small surface area.
High acoustic pressure and high acoustic intensity at low resonance frequency.
High efficiency, making the system requiring less driving power.
The use of low resonance frequency is required to avoid a high cavitation threshold, i.e., the intensity required to generate air bubbles within the stratum corneum of the patient's skin tissue. The cavitation threshold is inversely proportional to the frequency applied so the choice of a low resonance frequency of the transducer permits a lower acoustical pressure applied to the surface of the skin and transdermal drug delivery is effected
Reference is made to the following U.S. patents, which describe the “cymbal transducer design in greater detail:
   a) U.S. Pat. No. 4,999,819 Newnham, et al
   b) U.S. Pat. No. 5,276,657 Newnham, et al
   c) U.S. Pat. No. 5,729,077 Newnham, et al

### Design of Transducer Array

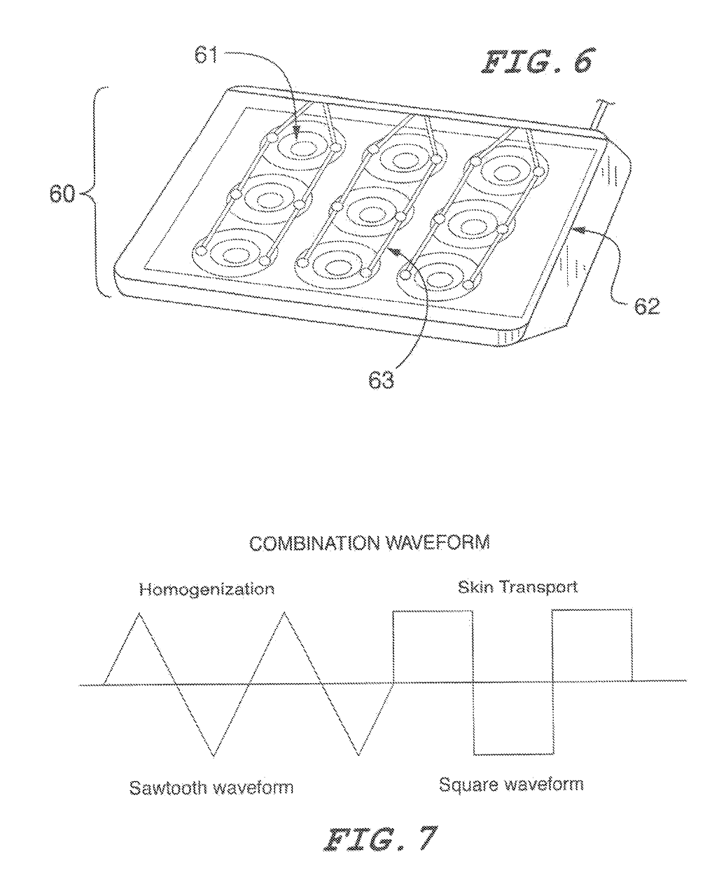

FIG. 6 shows an array 60 consisting of more than one-cymbal elements 61 arranged in an appropriate pattern onto a substructure or encased within a polymer housing 62. The cymbal elements 61 are connected in parallel by a series of electrical connections 63. Next the array 60 is then sealed in polymer potting material 62 composed of uralite, preferably. The array enables a portable, battery powered ultrasonic transmission, with sufficient power to effect drug delivery via a transdermal patch.

Mitragotri, Langler, Kost et al. In U.S. Pat. Nos. 4,767,402/4,780,212/5,814,599/5,947,921/6,002,961/6,018,678 and 6,041,253 discuss use of 225 mW/sq. cm. of intensity when using low frequency ultrasound. Applicant have discovered that the method applied by Mitragotri, et al envisioned application of ultrasonic drug delivery through just one skin delivery site. The concentration of energy at one site of the skin, through the stratum corneum, concentrates the ultrasonic transmission, leading to an increase in temperature rise within the fatty tissue within minutes. This has the effect of eventually closing the drug delivery pathway through the skin layer as the heat or cavitation energy alters the permeability of the stratum corneum layer or the epidermis layer of the skin, see FIG. 3, the structure of human skin. This is caused by repeatedly treating just one spot on the skin.

To avoid this problem the sonic applicator 1, as shown in FIG. 6, is designed to transmit ultrasonic signals through multiple transducers. In the preferred embodiment of this invention the transducers act in tandem, transmitting together. An alternate design could involve a transducer array whereby the activation of each element of the transducer array can be sequenced from transducer to transducer, possibly with different waveforms, frequency, amplitudes, and duty cycles between each transducer element. This has the affect of relieving the skin transport sites from continual ultrasonic stress and provides maximum variability in ultrasonic skin transport energy manipulation.

The transducer array as shown in FIG. 6 offers a means to spread out the drug pathway sites along the skin surface by providing ultrasonic transmission from the multiple transducer elements 61 of the array acting upon the skin. The transducer elements 61 may be activated simultaneously or sequentially to transmit ultrasound through the patch and through differing multiple sites on the skin surface. Additionally the frequency, intensity and waveform may be altered at each transducer element 61 within the array 60. This variation has the effect of increased efficiency, enhanced power utilization and lengthening the life span of the battery of the portable system. Additionally the alternating transducer elements 61 help to keep the drug homogenized within the B pocket of the transdermal patch and ensure that the skin is not overexposed to an excessive frequency of ultrasound.

An array of two or more transducers, especially the cymbal type, helps to push drugs through multiple skin transport sites. Moreover, the standard advantages of a transducer array reduce skin damage and improve the efficiency and transmitted acoustical intensity. By alternating the transducer activation sequence it is possible to avoid skin exertion and to assure greater longevity for the skin transport sites.

In the previous art the use of ultrasound coupled with iontophoresis, the application of electric currents applied to the skin, in various forms of drug delivery. In some instances ultrasound was used together with iontophoresis while in others ultrasound was a pre-treatment to the application of iontophoresis. Applicant have noted the method of iontophoresis and electroporation in combination with the apparatus of this invention could be used to enhance molecular transport through the skin.

The use of chemical substances, often referred to as chemical enhancers in the previous art, could enhance drug transport in this invention as well.

Pharmaceutical Substances Compatible with Ultrasonic Skin Transport

Apparatus and methods according to the present invention are useful for delivering a wide variety of medications to a patient. As described in greater detail herein below, the medication may be delivered transdermally, transcutaneously, intralumenally, and within solid tissue sites, where in all cases absorption of the medication or a pharmacologically active portion thereof into the underlying or surrounding tissue is phonophoretically enhanced by the application of ultrasonic or sonic energy. The medication may take any conventional form, including liquids, gels, porous reservoirs, inserts, or the like, and the medication or pharmacologically active portion thereof may be intended to treat or alleviate an existing condition or prophylactically prevent or inhibit another condition of the patient. The effect of the medication may be local, such as providing for anti-tumor treatment, or may be systemic. Suitable medicaments include broad classes of compounds normally delivered through the skin and other body surfaces or into solid tissues.

In general, such medication include or incorporate anti-invectives such as antibiotics and antiviral agents; analgesics and analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimatics; xanthine derivatives; cardiovascular preparations including potassium and calcium channel blockers, beta-blockers, and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers. By the method of the present invention, both ionized and nonionized drugs may be delivered, as can drugs of either high or low molecular weight.

Proteinaceous and polypeptide drugs represent a preferred class of drugs for use in conjunction with the presently disclosed and claimed invention. Such drugs cannot generally be administered orally in that they are often destroyed in the gastrointestinal tract or metabolized in the liver. Further, due to the high molecular weight of most polypeptide drugs, conventional transdermal delivery systems are not generally effective.

Common examples of pharmaceutical or nutritional compounds which could be contained within the modified transdermal patch of this invention include, but are not limited to: Acetaminophen, Antibiotics, Aspirin, Corticosterone, Erythromycin, Estradiol, Ibuprofen, Insulin, Nitroglycerin, and Nicotine, Steroids such as Progesterones, Estrogens, and Vitamins.

Any other pharmaceutical or nutritional compound approved for nutraceutical, medicinal or pharmaceutical use by the Food and Drug Administration of the United States of America may also be utilized. It is also desirable to use the method of the invention in conjunction with drugs to which the permeability of the skin is relatively low, or which give rise to a long lag-time. (Application of ultrasound as described herein has been found to significantly reduce the lag-time involved with the transdermal administration of most drugs).

Applicant have noted that most drugs are immersed within an excipient binder fluid such as saline or acetate composition to make them injectable. Insulin is often placed in acetate mixes. By altering the excipient solution it is possible to enhance and hasten skin transport and the homogenization effect within patch pocket B in conjunction with the application of ultrasound. Excipient solutions high in metallic or salt content for example can enhance the interaction between the drug and ultrasound. Applicant have noted that the effect of ultrasound at high intensity, or at low intensity but generating cavitation, could have a damaging effect upon many drug substances such as insulin whereupon the protein may become altered or damaged by excessive ultrasonic or cavitation frequencies and intensities. By choosing the correct excipient carrier solution the active drug substance may avoid damage and remain biofunctional after skin transport.

### Experiment 1: Fabrication of the “Cymbal” Transducer-Standard Construction

### Part List and Step by Step Manufacturing

### Parts List

### 1. Piezoelectric Ceramic

### Material:

PZT4 disc 0.5-inch diameter, 1-mm thickness (PKI402)

### SD 0.500-0.000-0.040-402

### Actual Supplier:

Piezo Kinetics Inc.

### Mill Road and Pine St.

### PO Box 756

### Bellefonte Pa. 16823

### 2. Titanium Caps

### Material:

Alfa Aesar, Titanium Foil, 0.25 mm thick, metal basis 5%, Item #10385

### Actual Supplier:

Alfa Aesar, A Johnson Matthey Company 30 Bond Street Ward Hill, Mass. 01835-8099, USA

### 3. Bonding Layer

### Material:

Eccobond 45LV+catalyst 15LV

### Actual Supplier:

Emerson & Cuming

### 46 Manning Road

### Billerica, Mass. 01821

### 4. Low Temperature Soldering

### Material:

Indalloy Solder #1E, 0.30″ dia×3 ft long

### Actual Supplier:

The indium corporation of America 1676 Lincoln AVE

### UTICA N.Y. 13502

### 5. Wires

### Material:

Stranded wire, Gauge/AWG: 30

Catalog number (Digikey): A3047B-100-ND

### Note: Maximum Temperature: 80 C

### Conductor Strand: 7/38

### Voltage Range: 300V

### Number of Conductors: 1

### Actual Supplier:

Alpha Wire Corporation

### 6. Housing Polymer

### Material:

Uralite FH 3550 part A/B

### Actual Supplier:

HB Fuller Company

### 7. Ethyl Alcohol

Note: 200 proof (at least)

### 8. Sand Paper

### Manufacturing Procedure: Step-by-Step

Reference is made to FIG. 4B:
1. Dye cut titanium foils into several disks. Materials: Titanium foil (2), circular saw 10.7 mm diameter.
2. Sand rough edges. One side of the disks results with edges. Those edges should be removed with sand (fine scale) paper. Materials: Sand paper (8)
3. Alcohol bath to remove dust generated by sanding the disks. Materials: alcohol (7)
4. Put disk into a high pressure (12000 torr) shaping tool (polished side up). For this step should be designed a custom-made punch dye in order to shape the disks into the dimensions reported in FIG. 2.
5. Sand rough edges again. Materials: sand paper (8)
6. Immerse in alcohol to remove dust. Materials: alcohol (7)
7. Wipe to remove alcohol and dust from disk
8. Measure thickness of cap with special measuring pen
9. Identify matching caps (by thickness). This step should be accurate because slight differences between the two caps generate spurious resonance into the cymbal.
10. Clean piezo disk ceramic with alcohol. Materials: piezodisks (1) and alcohol (7).
11. Screen printing on both sides with epoxy bond. Materials: bonding epoxy (3) and a tool for screen-printing (like T-shirt screen-printing). We should generate a ring of epoxy to glue the caps with the disks. This ring should be accurate and regular in order to avoid spurious resonance.
12. Place cymbals on ceramic disk
13. Place into a press. This press should just keep the cymbal made in place. It could be a custom-made tool where several cymbals are kept in place.
14. Place press into oven for at least 4 hours, 70 Celsius
15. Solder at maximum 270 Celsius at 4 points per piece. Materials: wires (5) and low temperature solder (4)

The transducer produced by the above procedure is a standard construction. To form a Stacked Construction transducer two or more transducers are placed directly atop one another as shown in FIG. 4C and fitted together. To form an array the transducers are generally connected in parallel electrically within the polymer or epoxy bonding material as shown in FIG. 6, in either single element form or in a stacked construction format.

### Experiment 2

### Testing of Cymbal Transducer, Single Element

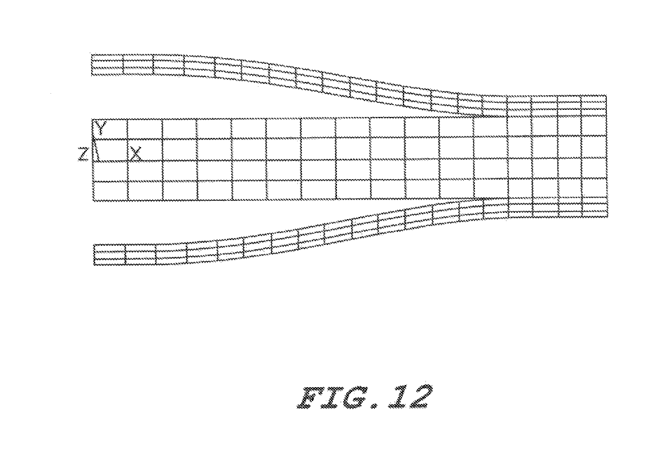

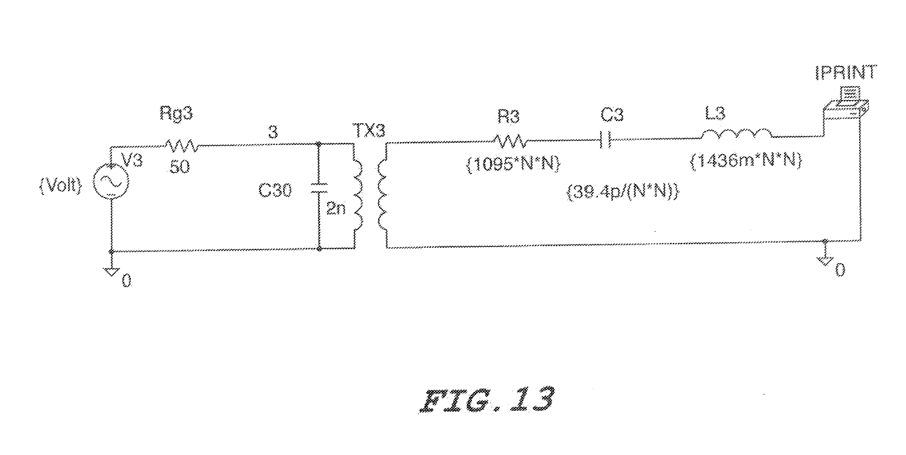

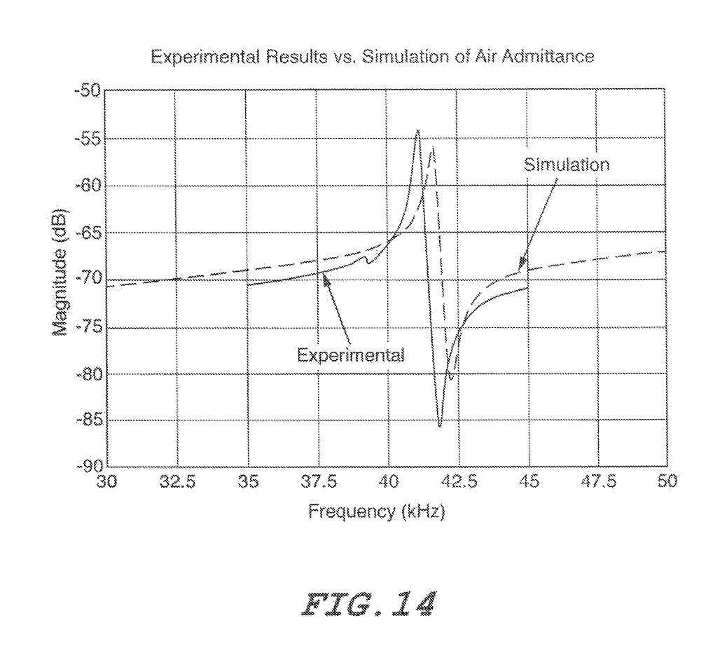

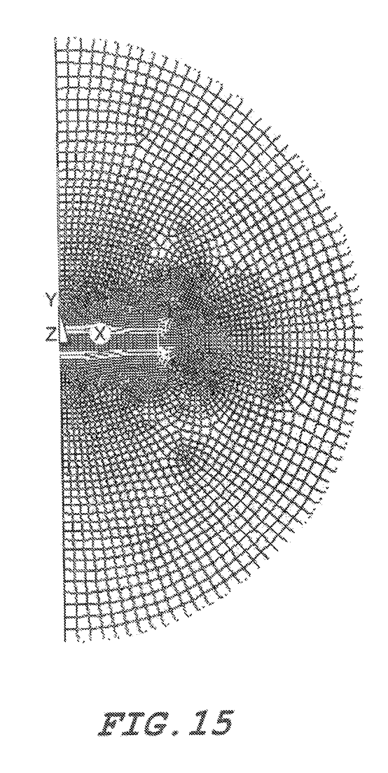

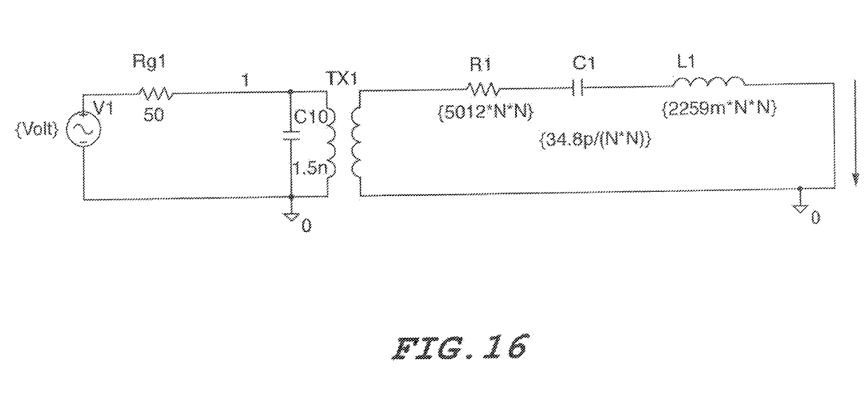

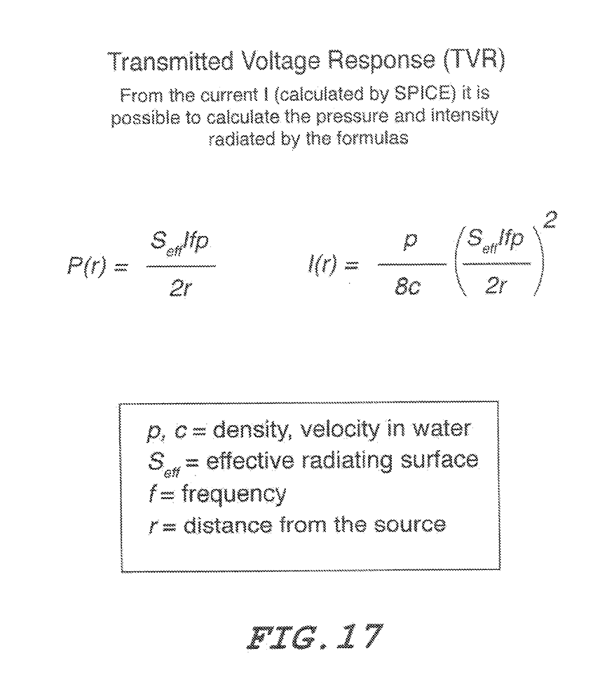

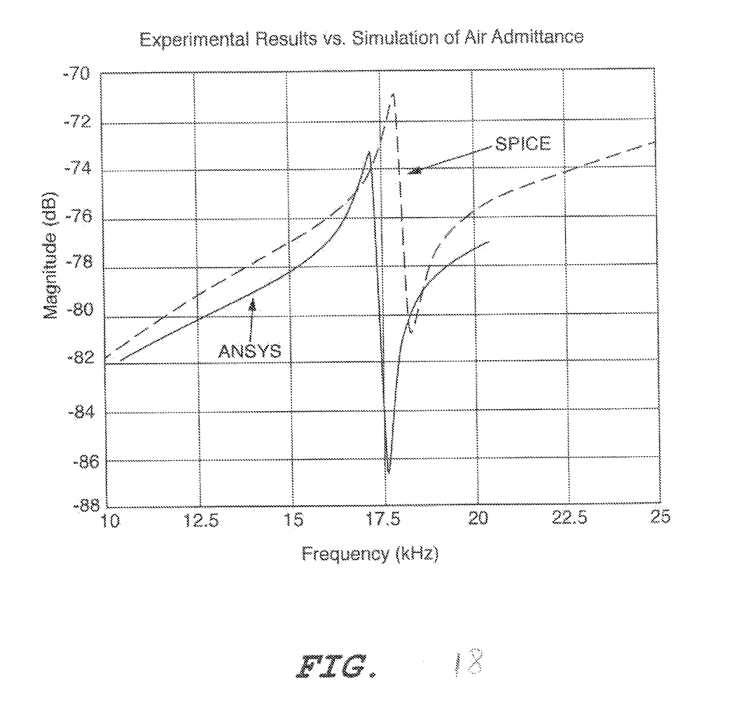

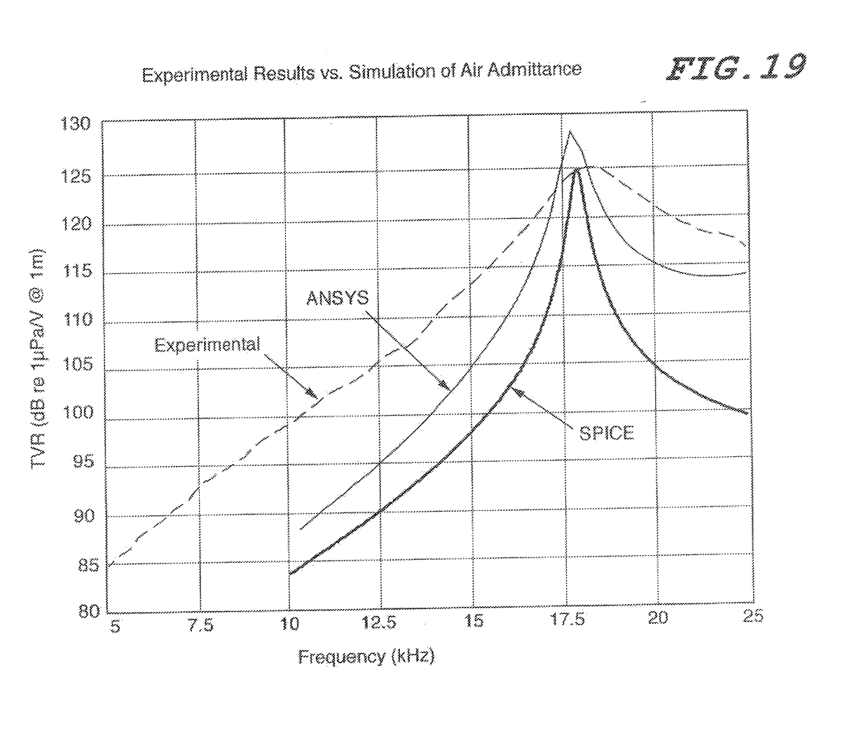

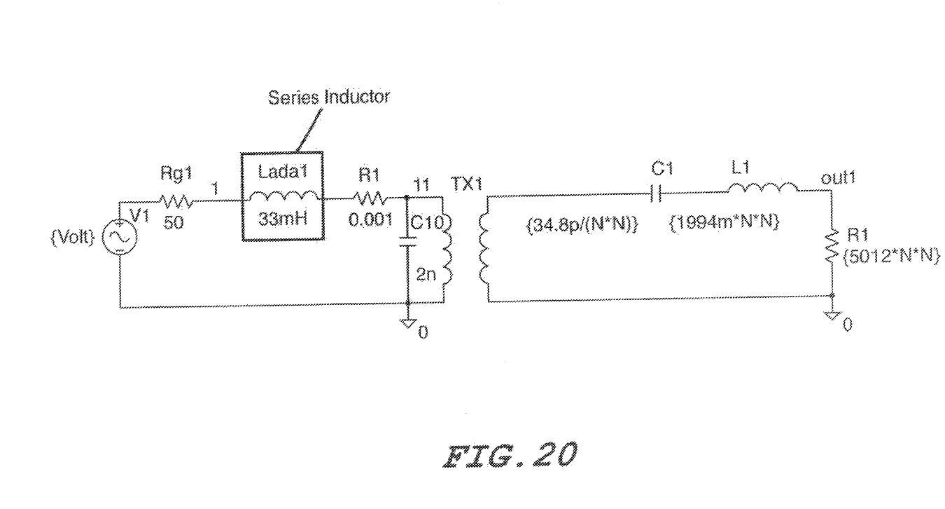

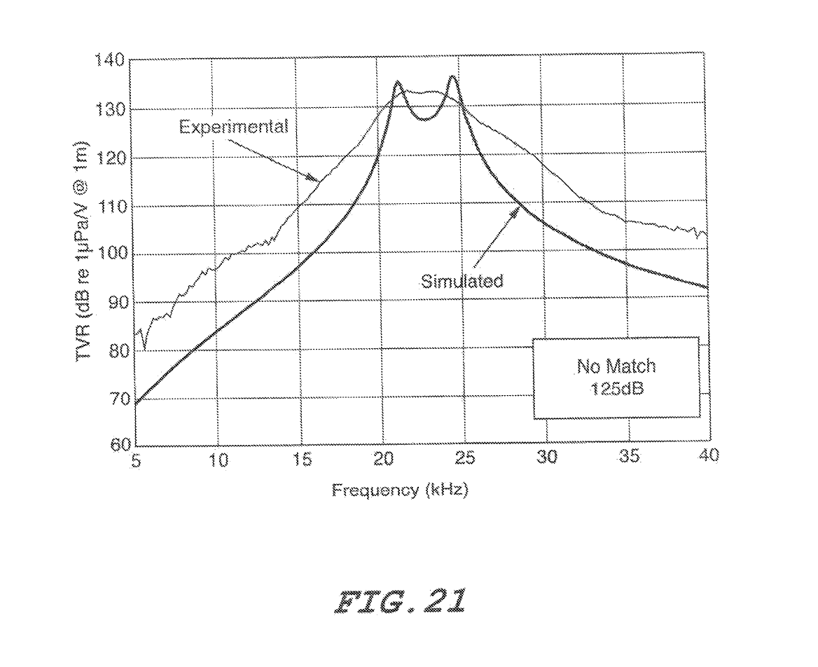

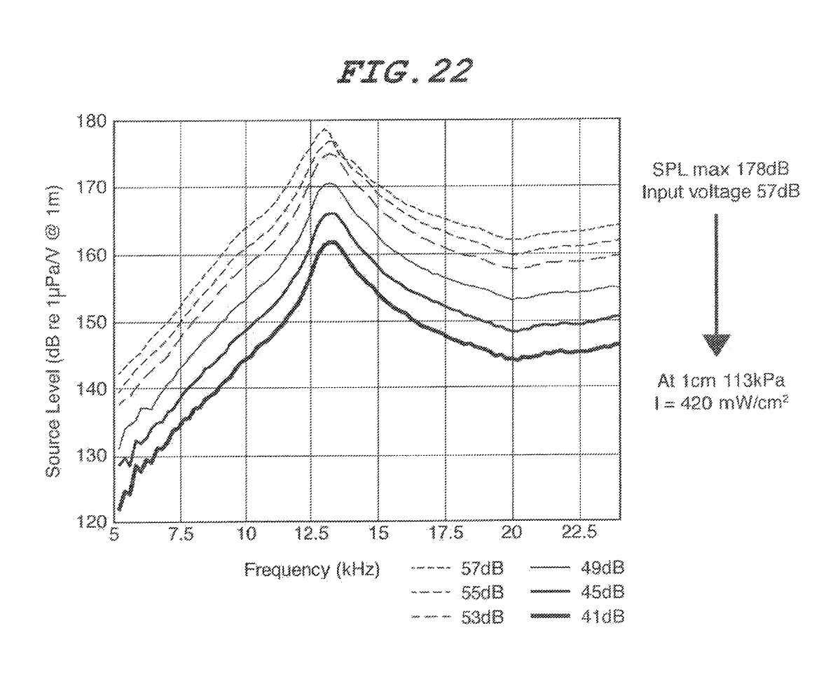

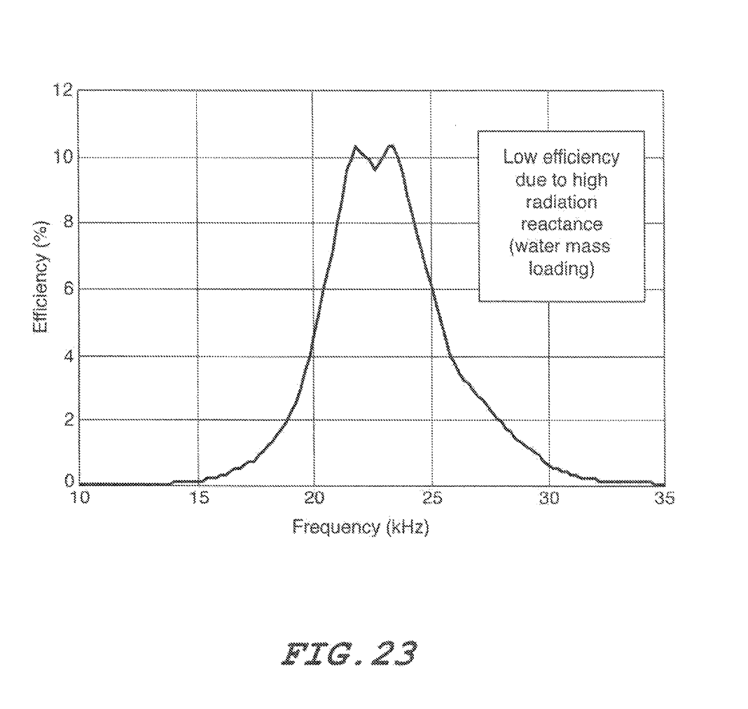

A series of physical tests were conducted on the single element cymbal transducer fabricated according to the steps of Experiment 1, using standard analysis procedures common to the ultrasonic and transducer industry. The results are illustrated in FIGS. 12 through 23 and show that the single element transducer is a highly efficient system producing an ultrasonic transmission within two ranges:

### Range—A

| | |
|---|---|
| TRANSDUCER TYPE | Single element “Cymbal” design |
| FREQUENCY | 20k Hz |
| INTENSITY: LOWEST SETTING | 125 mW/sq. cm. |
| DESIGN | Standard Construction |

### Range—B

| | |
|---|---|
| TRANSDUCER TYPE | Single element “Cymbal” design |
| FREQUENCY | 20k Hz |
| INTENSITY: LOWEST SETTING | 225 mW/sq. cm. |
| DESIGN | Stacked Construction |

### Experiment 3

### Testing of Transducer Array Consisting of Nine Single Cymbal Elements Connected in Parallel as Illustrated in FIG. 6

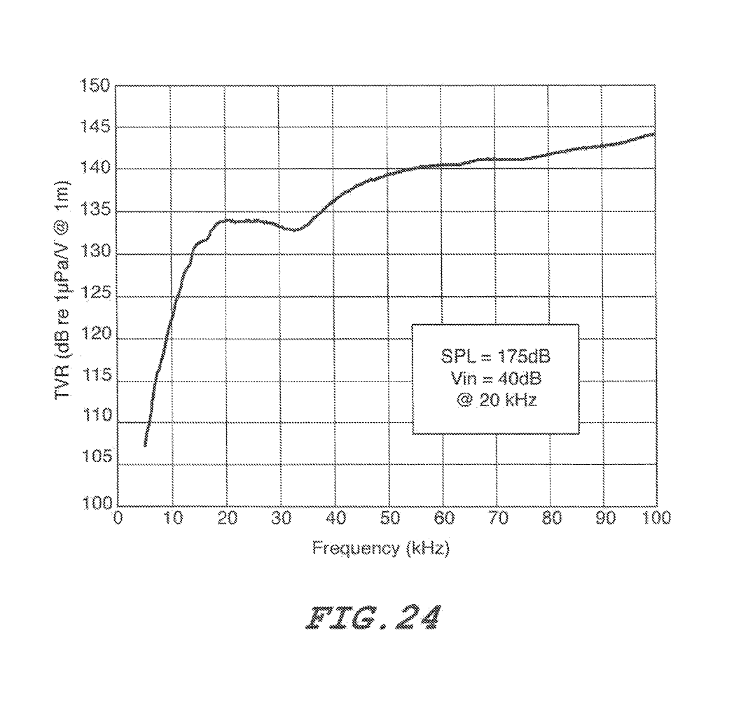

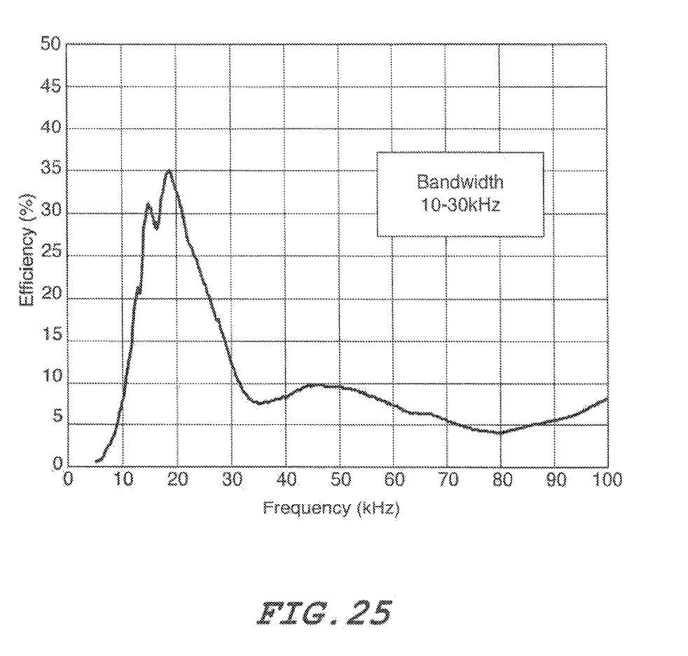

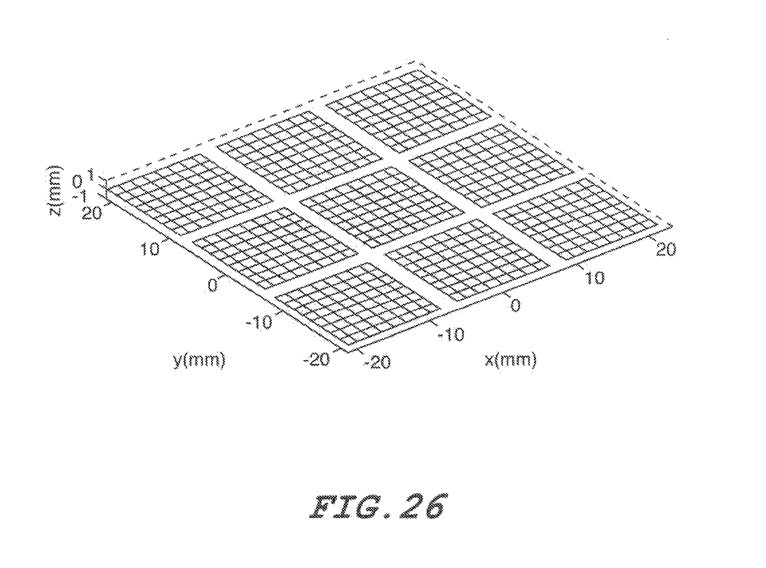

A series of physical tests were conducted on the single element cymbal transducer fabricated according to the steps of Experiment 1, using standard analysis procedures common to the ultrasonic and transducer industry. The results are illustrated in FIGS. 24 through 28 and show that the single element transducer is a highly efficient system producing ultrasonic transmission within two ranges:

### Range—A

| | |
|---|---|
| TRANSDUCER TYPE | Single element “Cymbal” design |
| FREQUENCY | 20k Hz |
| INTENSITY: LOWEST SETTING | 125 mW/sq. cm. |
| DESIGN | Standard Construction using nine |
| | elements |

### Range—B

| | |
|---|---|
| TRANSDUCER TYPE | Single element “Cymbal” design |
| FREQUENCY | 20k Hz |
| INTENSITY: LOWEST SETTING | 225 mW/sq. cm. |
| DESIGN | Stacked Construction using nine |
| | elements |

Applicant theorize that arrays with different orientation of cymbals and with combinations of standard and stacked arrays can be used to increase efficiencies and to improve the effective delivery of drugs.

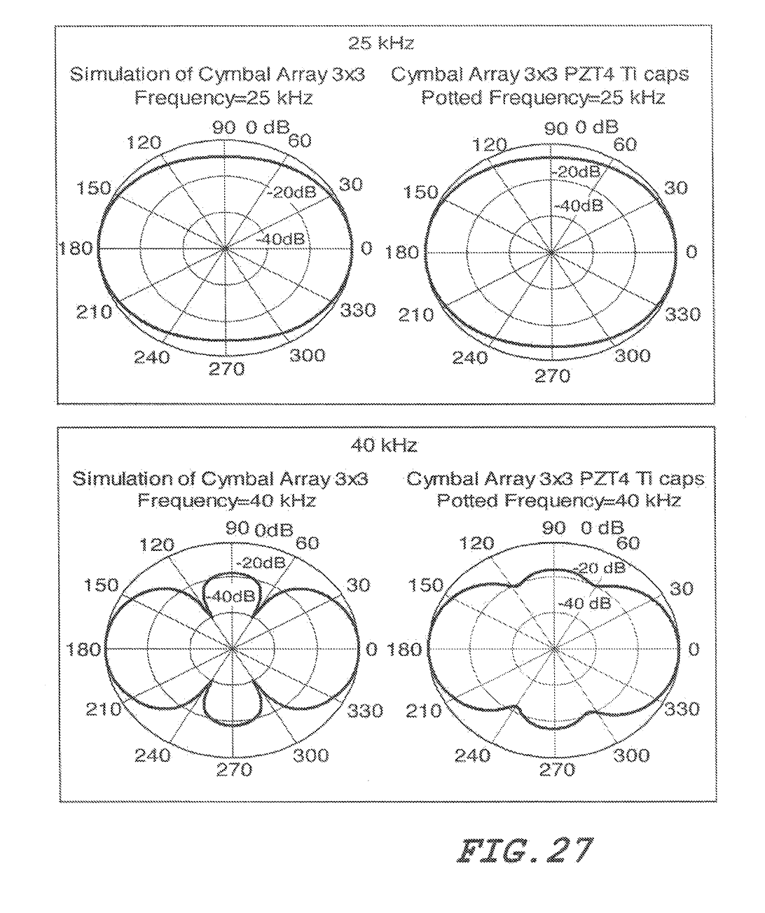

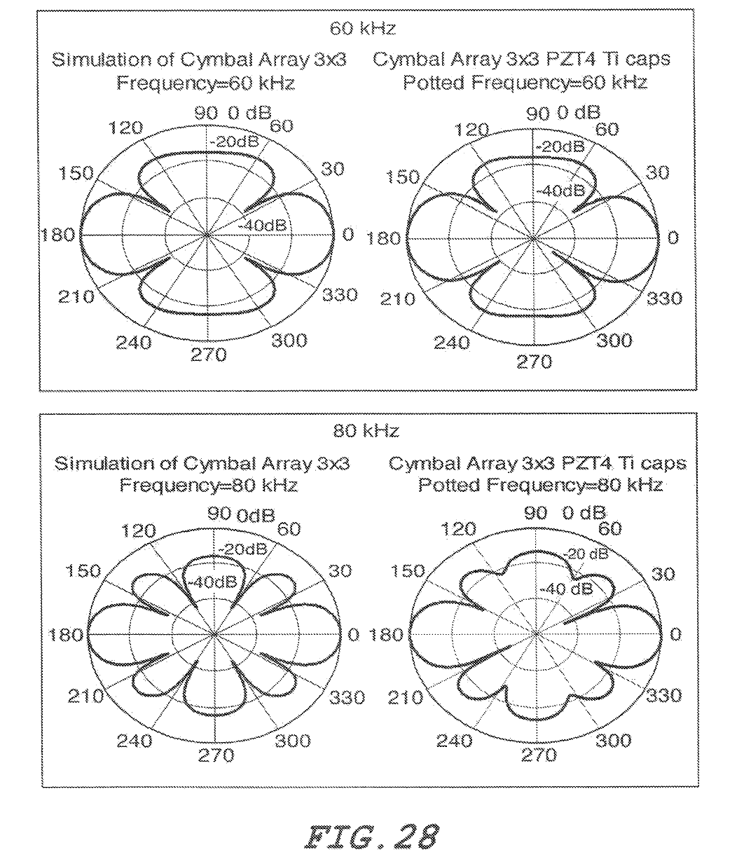

FIGS. 27 and 28 illustrate that it is possible to obtain alternating frequency outputs from the transducer array. In tests the array using nine—single elements in a standard construction and in a stacked construction produced frequency outputs, which could be varied from 20 kHz to 100 kHz. In FIG. 27 it can be seen that the ultrasonic transmissions were most uniform at the lower frequency range. In FIG. 27 it can be seen that the ultrasonic transmissions were irregular at the higher frequency range. In all case the transducers could be made to emit a purely sinusoidal waveform or be converted to a combination waveform consisting of sawtooth and square waves as illustrated in FIG. 7. In the above mentioned tests the ultrasonic driver circuit, the frequency generator, was set to propagate 100 milliseconds of sawtooth waveform followed immediately by 100 milliseconds of square waveform, before re-cycling back to sawtooth waveform.

### Experiment 4

### Testing of Transducer Array Consisting of Nine Single Cymbal Elements Connected in Parallel as Illustrated in FIG. 6 for Power Utilization

The transducer arrays according to Experiment 3 were tested for power utilization. The transducers mentioned in the prior art, specifically U.S. Pat. No. 4,999,819 Newnham, et al; U.S. Pat. No. 5,276,657 Newnham, et al and U.S. Pat. No. 5,729,077 Newnham, et al, required significant power to drive the transducers to generate an ultrasonic transmission. In this invention the transducers, whether configured in a single element or as an array, in either a standard or stacked construction, needed to operate using low power. The portable nature of the final drug delivery device, as depicted in FIGS. 1 and 2, required a system, which is worn by the patient.

Accordingly a portable power source, ideally a rechargeable battery, would be required to drive the ultrasonic system. As a result the objectives of this invention with regards to power utilization include:
1. Low Power requirement to drive the transducer, ideally in the form of standard commercially available battery sources.
2. Long duration power, providing at least one full day of continuous power.

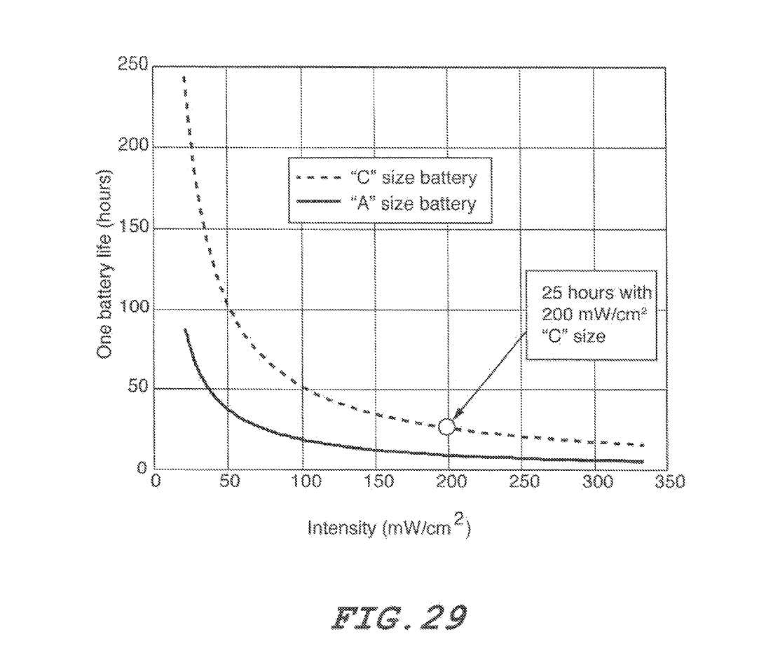

Tests were conducted using the standard nine element “Cymbal” design array set to operate at 20 k Hz frequency and at varying intensity levels, powered by a standard “A” or “C” type battery. The results are illustrated in the graph of FIG. 29, showing that a useful power life of 25 hours was obtained at an intensity level of 200 mW/sq. cm. with continued constant usage to the transducer array.

As a result a significant milestone in transducer design was achieved, wherein the transducers were fabricated to enable battery power to drive the ultrasonic signal and the efficiency of the power utilization of the transducers were demonstrated to provide a low battery drain rate, thereby extending the life of the power source. Accordingly a portable or wearable ultrasonic drug delivery system employing ultrasonic drug delivery is possible utilizing conventional battery sources coupled with the transducers of this invention.

### EXPERIMENT 5

### Testing of Transducer Effect Upon Active Pharmaceutical Substance Drug Survivability after Ultrasonic Exposure

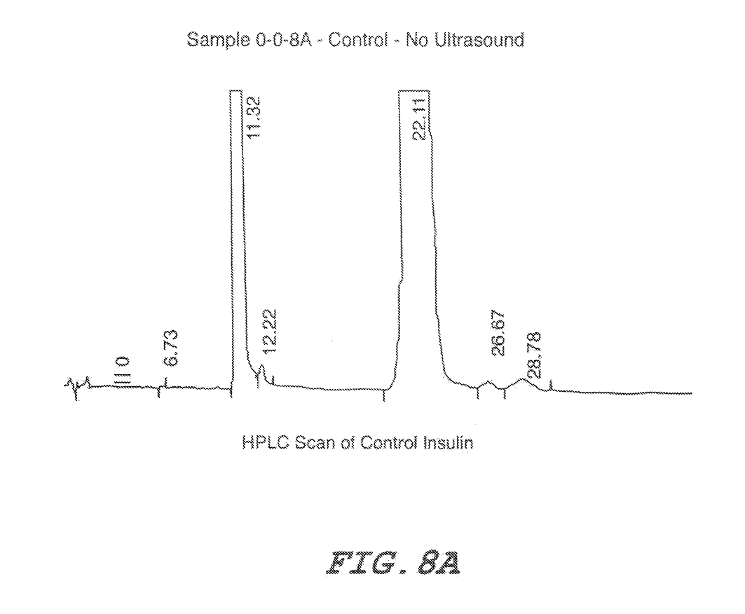

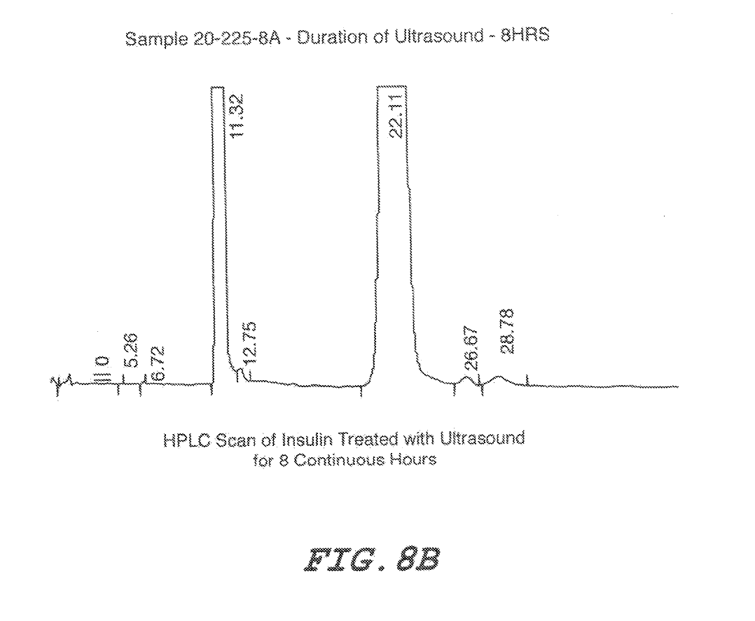

Since the transducers of this invention are designed primarily for application in a drug delivery system the Applicant deemed it necessary to test the effect of the ultrasonic signal upon an active pharmaceutical substance. High intensity and high frequency ultrasound is theorized to be capable of inducing a cavitation effect within a drug, leading to an increase in temperature and a degradation of the drug molecule. For the following experiment Insulin (Humulin Regular-supplied by the Eli Lilly Company) was subjected to ultrasound emitted from a stacked array of the transducers of this invention, set to operate at 20 k Hz frequency and at 125 mW/sq. cm intensity level, for one, eight and eleven continuous hours of exposure. The insulin was placed in a plastic pouch within a hydrophone tank containing water and stirred during ultrasonic exposure. A control sample, which was untreated, but allowed to sit in the pouch and tank for one, eight and eleven hours, was also made. Samples were sent to Celsis Laboratories for independent analysis. All samples showed no change in the insulin from the untreated insulin. FIG. 8A shows the HPLC scan of the control sample, showing no degradation of either the insulin or the excipient solution of the Humulin Regular sample. FIG. 8B shows the HPLC scan of the ultrasonically treated eight hour sample, showing that there also was no degradation of either the insulin or the excipient solution of the ultrasonically treated Humulin Regular sample, even after eight hours of continuous exposure.

Accordingly there appears to be no damage caused to the insulin molecule as a result of following ultrasonic transmission factors associated with transducers of this invention:
Low frequency
Low Intensity
Alternating waveform

### Experiment 6

### Testing of Transducer Capability to Delivery an Active Pharmaceutical Substance in a Live Animal Model Using a Ultrasonically Enhanced Transdermal Drug Delivery Technique

A four-element transducer was fabricated using four standard cymbal element transducers in one array system (Array #1) and four stacked cymbal element transducers in another system (Array #2). Array #1, the standard array, was set to operate at 20 k Hz frequency and at 125 mW/sq. cm intensity level. Array #2, the stacked array, was set to operate at 20 k Hz frequency and at 225 mW/sq. cm intensity level.

The transducer's arrays were fitted with a reservoir at the bottom end, into which Humulin Regular Insulin (supplied by Eli Lilly Company) was inserted. A total of 100 cc of insulin was added, providing 100 units of insulin for the each test.

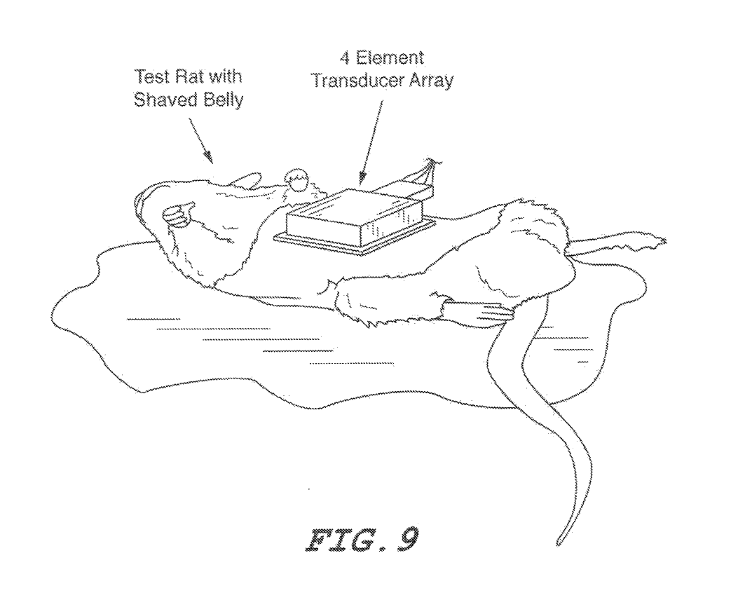

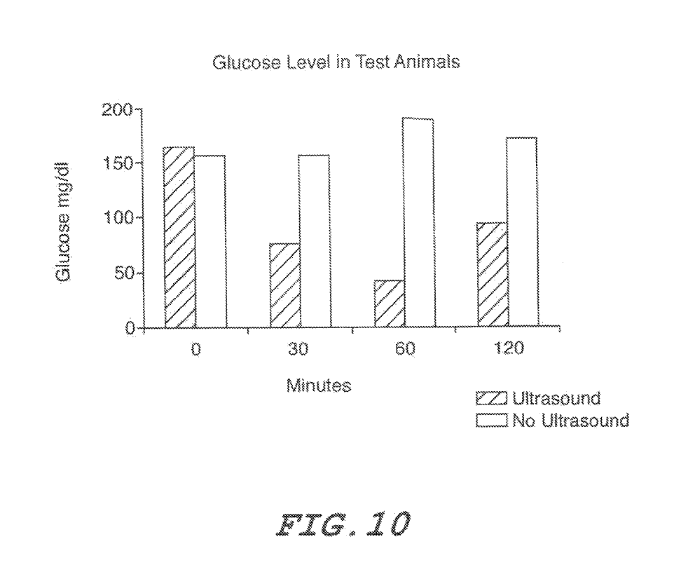

Ten test rats were assembled and anesthetized, as seen in FIG. 9. The belly of the rats were shaved to produce a skin area as close in configuration as would be present in the human situation. The transducer arrays were placed directly onto the rat skin surface and adhered to the skin by means of an adhesive.

Two groups of test rates were assembled. The first group (Group-1) would receive the ultrasonic transmission while the second group would receive no ultrasound. In the second group (Group-2) the transducers arrays were loaded with insulin and the insulin was allowed to pool onto the surface of the rat skin, but there was no active ultrasonic transmission.

Next a frequency generator was employed to propagate the pulsed ultrasonic transmission, which used 100 millisecond pulses, with a pulse rate of one pulse per second, a duty cycle of 10%, for one hour.

Both Group-1 and Group-2 animals were tested for 120 minutes. Blood samples were taken from the animals according to standard investigative procedure every 30 minutes for the first hour and every hour after and analyzed for glucose levels and the presence of insulin. The Group-1 animals were exposed to ultrasound for 60 minutes, after which the ultrasound was terminated for the balance of the test period. Glucose levels in both groups were observed over the 120 minute period.

FIG. 10 illustrates the results of these tests, with an average of the data compiled across the number of tests conducted. Specifically this data relates the average Glucose level of the Group-1 animals treated with ultrasound and the Group-2 animals, which were untreated, but where the insulin was placed in a blank array (containing no transducers) and placed upon the skin surface. At minute 0, before the ultrasound was activated, both groups had similar starting glucose levels. At minute 30 and minute 60 the Group-1 animals showed a significant reduction in glucose levels while the Group-2 animals showed no lowering in glucose level. At minute 60, the ultrasound was terminated and the animals monitored for another 60 minute period. The Group-2 animals showed no decrease in glucose levels, as the insulin was not absorbed through the skin. The Group-1 animals, which had a lowering of their glucose levels during active ultrasound transmission, indicating that the ultrasound enabled the permeation of the insulin through the skin, were observed to have a rise in their glucose levels upon termination of the ultrasound.

At minute-120 the Group-2 animals showed no decrease while the Group-1 animals showed their glucose levels to be rising to the previous-pre-ultrasound levels.

This test showed that the insulin was only permeated through the skin via the ultrasound emitted from the transducer arrays, and only with the presence of active ultrasound. The tests also confirmed that insulin, placed on the skin or delivered via a transdermal patch will not permeate through the skin on its own. These tests also confirm the validity of the transducer designs of this invention as effective means for delivering ultrasonically enhanced transdermal drug delivery. These tests also showed that insulin delivered transdermally by the portable transducers can effectively decrease glucose levels. This result showed that the insulin is not only absorbed through the stratum corneum but it is also absorbed into the blood stream in an effective form and can cause its metabolic effect of lowering glucose.

FIG. 11 shows a test rat after the ultrasonic exposure. A section of the skin where the transducer was placed is marked on the skin. Examination of the skin features showed no damage to the skin surface, no discoloration or abnormal fractures after the application of ultrasound emitted from the transducers of this invention.

### CONCLUSION

The device of this invention is intended to provide certain key drug delivery functions:
1. Non-Invasive drug delivery through his use of ultrasound applied transdermally to a patient's skin surface.
2. Penetration/absorption enhancement through the skin so that medicines contained within a Transdermal Patch will become more readily absorbed through the skin layers into the patient's blood stream.
3. Homogenization and droplet size reduction of pharmaceutical agents contained within a Transdermal Patch, to make the resulting ultrasonically treated drug more readily absorbable through the patient's skin layers. This may be especially suited to difficult to administer drugs such as insulin and various hormone medicines.
4. The device is intended to go with the patient, to be wearable by the patient, containing rechargeable batteries to provide treatment mobility.

Key elements of this invention, which distinguish it over the prior art, include:
a) The ability to provide for a portable and wearable ultrasonic drug delivery device, which goes with the patient. U.S. Pat. No. 4,767,402, Kost, et al, describes a method of ultrasonic drug delivery, which is not portable and applicable to a clinical setting. As such it and many of the related patents are not practical as the patient may have to be treated over several hours of therapy.
b) The drug delivery pathway includes hair follicle and skin pore delivery means as opposed to breaching the stratum corneum. The drug contained within the drug pocket of the Transdermal Patch ultimately penetrates into the patient's blood stream, aided by the sonic transmission through the skin pores or hair follicles and into the muscular of the patient. This pathway approach reduces the chance of damaging the skin and enables the use of lower ultrasonic frequencies and intensities. Methods taught in the prior art were found to possess higher frequency and intensity levels, which offered the possibility of damaging the active pharmaceutical substance during ultrasonic drug delivery. Damaging the drug could result in the conversion of the drug to a toxic compound.
c) Key to this invention is the use of a transducer array, which enables ultrasonic skin transport at more than one site on the skin, thereby assuring a greater chance of effective skin transport and avoiding overtaxing just one delivery site. The use of multiple transducers offers varied treatment effects to facilitate maximum skin transport of the target active pharmaceutical agent, by providing tandem drug transport across multiple transducer elements, by enabling sequencing of the transducer elements in the array, whereupon the transducers may act at different frequencies and intensity levels of ultrasound.
d) Using an array of transducers in a portable, wearable ultrasonic drug delivery device, especially utilizing cymbal type transducers, provides higher power utilization efficiencies and helps to avoid the damaging effects of excessive cavitation upon the skin. The array makes possible long duration battery supplies providing sufficient power to enable the apparatus to function for several days between recharge or replacement cycles. The use of a rechargeable battery supply, ideally with batteries contained with the strap of the device, afford total mobility for the patient and a reliable power supply for the device over several months of recycled use.
e) Applicant note that the use of transmission in both the sonic and ultrasonic ranges may need to be combined to achieve optimal transport through the skin or mucosal membranes.
f) To be a wearable device it is essential to deliver the proper dose of a drug across the skin, in minutes as opposed to the hours noted in the previous art.
g) Such advantages are part of the invention as described herein:
h) The use of low frequency ultrasound, ideally from 20-100 kHz, which uses alternating waveform (from sawtooth to square wave), with cymbal type transducers, which enable battery power ultrasonic transmission. A transducer array to avoid over exerting a single skin transport site and providing versatility in ultrasonic frequency and intensity ranges per transducer element.
1. A method for conducting the transport of active pharmaceutical compositions through the body surface of an individual, comprising applying ultrasound through a transdermal delivery device which is affixed to a portable, programmable ultrasonic regulator device, which itself is worn by the individual wherein said ultrasound is applied at an intensity and for a time period effective to enable movement of a therapeutic quantity of said active pharmaceutical composition from the transdermal delivery device through the skin, for the purpose of effecting regulated, and timed drug delivery to the individual.
2. The method of claim 1, wherein said ultrasound has a frequency in the range of about 20 kHz to 10 MHz.
3. The method of claim 1, wherein said intensity of said ultrasound is in the range of about 0.01 W/cm.sup.2 to 5.0 W/cm.sup.2.
4. The method of claim 1, wherein the wearable, portable sonic device is affixed onto or connects to a transdermal patch which provides the transdermal delivery of drugs to the individual.
5. The method of claim 1 wherein rechargeable batteries are used as the primary battery supply.
6. The method of claim 1, wherein the wearable, portable sonic device is controllable through programmable settings such as to the quantity of drug released by the device, the time interval of active ultrasonic drug delivery, the time interval between ultrasonic drug delivery, the frequency and intensity of the ultrasonic signal, the basal delivery schedule of drug dosing and the bolus delivery schedule of booster doses of a particular drug, with both automatic functions and a manual operation capability.
7. The method of claim 1, wherein the ultrasound is applied continuously.
8. The method of claim 1, wherein the ultrasound is pulsed.
9. A method for enhancing the delivery of a pharmaceutical compound through the skin of a patient, through the use of a wearable, portable and programmable ultrasonic drug delivery system, said method comprising:
   applying the pharmaceutical compound to the body surface;
   passing an electrical signal through the skin of the patient; and
   Transmitting vibrational energy from said to the wearable, portable and programmable ultrasonic drug delivery system through a transdermal patch containing said pharmaceutical compound to the body surface to enhance penetration of the pharmaceutical compound into the tissue surface.
10. A method as in claim 9, wherein the body surface is skin and the drug delivery pathway is through the pores and hair follicles of the skin.
11. A method as in claim 9, wherein the electrical signal is generated within the enclosure of the a wearable, portable and programmable ultrasonic drug delivery system, and said active pharmaceutical substance is supplied by means of a modified transdermal patch.
12. A method as in claim 9, wherein the electrical signal is generated externally of the patient and delivered transcutaneously to the wearable, portable and programmable ultrasonic drug delivery system
13. A method as in claim 9, wherein the electrical signal has a voltage in the range from 1V to 20V.
14. An assembly for the transdermal administration of a drug to a patient, comprising: a base unit of a wearable, portable and programmable ultrasonic drug delivery system having a timer and electrical connections for issuing electronic timing information from said timer; a transdermal patch unit containing transducers in one pocket of the patch and active pharmaceutical substance in another pocket, said transdermal patch unit electrically connected to said base unit, said transdermal patch unit having a housing defining a space therein for receiving a drug and said housing having drug dispensing conduit means formed therein, a skin-contacting surface to be placed on a patient's skin, dispensing means for selectively causing time-dependent dispensing of a drug from said space in said housing through said conduit means to said skin-contacting surface and to the patient's skin, and means for generating pressure waves at said skin contacting surface for facilitating transdermal absorption of the drug dispensed to said skin contacting surface.
15. The assembly according to claim 14, wherein said dispensing means is a transdermal patch comprising a semi-permeable membrane between the patch and the patients skin surface providing an on-off drug delivery valving mode, wherein the “valve” is open upon the application of active ultrasound and the valve is closed when the ultrasound is discontinued, preserving the drug for active delivery cycles only.
16. The assembly according to claim 14, wherein said pressure wave generating means comprise a transducer or array of transducers contained within the transdermal patch and circuit means operatively connected to said transducer(s) for driving said transducer(s) at a given frequency and intensity level.
17. The assembly according to claim 14, wherein said circuit means comprises wave form generator means for generating an electronic signal formed of a sawtooth wave in a frequency range of between 20 kHz and 100 kHz and of a square wave superimposed thereon in a frequency range of between 20 kHz and 100 kHz.
18. The assembly according to claim 14, wherein said pressure waves generating means include an ultrasonic transducer for generating ultrasonic waves aimed at the patient's skin, by means of transducers which are contained within a transdermal patch and an ultrasonic waveform generator drivingly connected to said transducer.

### Hair Follicle Transporting

19. A means of ultrasonic drug delivery employing a programmable, wearable and therefore portable ultrasonic applicator device which is affixed to a transdermal drug delivery patch, wherein a strap which is used to hold the device onto the body of the patient also acts to constrict and apply pressure to the skin section directly under the transdermal patch for the purpose of enhancing the effect of ultrasonic drug transport through the skin pore and hair follicle drug pathways within the structure of the skin, leading to direct drug deposition within the bloodstream of the patient.
20. Apparatus as in claim 19 wherein transducer assembly is composed of a single cymbal type ultrasonic transducer.
21. Apparatus as in claim 19 wherein transducer assembly is composed of an array of transducers.
22. An apparatus as claimed in claim 19 wherein cymbal type ultrasonic transducers are employed in said array.

### Multiple Transport Sites

23. A means of conveying ultrasonic drug delivery through the use of an array of transducers, employed to deliver ultrasonic energy through a transdermal patch, wherein the array makes possible the application of the ultrasonic drug transport through a number of multiple skin transport sites, for the purpose of avoiding premature damage to the skin transport sites and effecting the greatest quantity of deliverable drug from the patch, through the patients skin and into the bloodstream.
24. A means of conveying ultrasonic drug delivery as claimed in claim 23, wherein the transducer array is comprised of cymbal type transducers, number more than one transducer element.
25. A means of conveying ultrasonic drug delivery as claimed in claim 23, wherein the multiple transducer elements transmit ultrasound at identical frequencies and intensity levels to each other.
26. A means of conveying ultrasonic drug delivery as claimed in claim 23, wherein the multiple transducer elements transmit ultrasound at differing frequencies and intensity levels to each other.

### Sawtooth to Square Wave Form

27. A means of enhanced ultrasonic drug delivery employing an transducers and oscillatory system which will impart a square waveform through a transdermal patch and through the outer layers of the skin of a patient wearing said transdermal patch wherein the square wave form enables effective skin transport while also avoiding excessive cavitation energies to the skin which could damage the skin tissue.
28. A means of enhanced ultrasonic drug delivery employing an transducers and oscillatory system which will impart a sawtooth waveform through a transdermal patch and through the outer layers of the skin of a patient wearing said transdermal patch wherein the square wave form enables effective skin transport, while also avoiding excessive cavitation energies within the stored pharmaceutical agent contained within said patch, which otherwise could damage the active pharmaceutical.
29. A cymbal type ultrasonic transducer, suitable for use in ultrasonic drug delivery, which is minute in size and weight and provides ultrasonic transmissions in conjunction with the appropriate oscillatory drive assembly, producing a sonic transmission which alternates periodically from a sawtooth to square wave form.
30. A means of enhanced ultrasonic drug delivery employing an transducers and oscillatory system which will impart a sawtooth waveform through a transdermal patch for the purpose of homogenizing pharmaceutical substances within the patch into smaller beadlets suitable for effective skin transport.

### Single Element Transducer

31. An ultrasonic transducer assembly suitable for use in ultrasonic drug delivery applications employing a single cymbal type ultrasonic transducer design according to the type disclosed in U.S. Pat. No. 4,999,819 Newnham, et al; U.S. Pat. No. 5,276,657 Newnham, et al and U.S. Pat. No. 5,729,077 Newnham, et al.
32. An apparatus as claimed in claim 31 wherein cymbal type ultrasonic transducers are employed in a transducer assembly array consisting of more than one transducer element.

### Single Element Transducer-Stacked Construction

33. An ultrasonic transducer assembly suitable for use in ultrasonic drug delivery applications employing a stacked configuration cymbal type ultrasonic transducer design according to the type disclosed in U.S. Pat. No. 5,729,077 Newnham, et al, WHEREIN TWO OR MORE Transducers are stacked on top of one another for the purpose of increasing the intensity of an ultrasonic transmission while also increasing the power efficiency of the transducer system as a whole.
34. An apparatus as claimed in claim 33 wherein said stacked cymbal type ultrasonic transducers are employed in a transducer assembly array consisting of more than one transducer element.
35. A means of conveying ultrasonic drug delivery through the use of an array of transducers, employed to deliver ultrasonic energy through a transdermal patch, wherein the array makes possible the application of the ultrasonic drug transport through a number of multiple skin transport sites, for the purpose of avoiding premature damage to the skin transport sites and effecting the greatest quantity of deliverable drug from the patch, through the patients skin and into the bloodstream.
36. A means of conveying ultrasonic drug delivery as claimed in claim 35, wherein the multiple transducer elements transmit ultrasound at identical frequencies and intensity levels to each other.
37. A means of conveying ultrasonic drug delivery as claimed in claim 35, wherein the multiple transducer elements transmit ultrasound at differing frequencies and intensity levels to each other.
38. A means of effecting the ultrasonic transdermal delivery of drugs employing a transducer assembly wherein said transducer is a class V flextensional transducer “cymbal” type electrically connected with an ultrasonic waveform generator.
39. The assembly according to Claim 38, wherein said transducer is an array consisting of several class V flextensional transducers, cymbal type elements electrically connected with said ultrasonic waveform generator.
40. The assembly according to Claim 38, wherein said transducer is an array consisting of several class V flextensional transducers, cymbal type elements, assembled in a stacked configuration and electrically connected with said ultrasonic waveform generator.
41. The assembly according to claim 38, including a microprocessor means for controlling at least one basal timing sequence and at least one bolus timing sequence for drug delivery.
42. The assembly according to claim 38, wherein said transducer is a planar piezo-electric disc electrically connected with said ultrasonic waveform generator.
43. The assembly according to claim 39, wherein said transducer array is composed of multiple planar piezo-electric discs, cymbal type, electrically connected with said ultrasonic waveform generator.
44. The assembly according to claim 40, wherein said transducer is an array consisting of several planar piezo-electric disc, cymbal type elements assembled in a stacked configuration and electrically connected with said ultrasonic waveform generator.
45. A means of effecting the ultrasonic transdermal delivery of drugs employing a transducer assembly wherein said transducer is a class V flextensional transducer “cymbal” type electrically connected with an ultrasonic waveform generator, the transducer assembly including in said ultrasonic waveform generator at least one sine wave generator, at least one square wave generator, a summing circuit having respective inputs connected to said sine wave generator and to said square wave generator for generating a superimposed signal of said sine wave and said square wave connected to said transducer or transducer elements in an array of transducers contained within a transdermal drug delivery patch.
46. The assembly according to claim 45, wherein said timing circuit is operative for activating said waveform generator in alternating on and off states in programmed sequence.
47. The assembly according to claim 45, including in said timing circuit EEPROM means for storing at least one timing program for timing said programmed sequence.
48. The assembly according to claim 45, including stored in said EEPROM means at least one basal timing sequence and at least one bolus timing sequence for drug delivery.
49. The method of claim 45 wherein the ultrasound is applied continuously.
50. The method of claim 45 wherein the ultrasound is pulsed.

### Transducers Built into Transdermal Patch

51. A means of enhanced ultrasonic drug delivery employing transdermal patches, which are excited by ultrasonic oscillatory circuits and transducers, contained directly within the patch.

Having described the invention in the above detail, those skilled in the art will recognize that there are a number of variations to the design and functionality for the device, but such variations of the design and functionality are intended to fall within the present disclosure.

1. A method for conducting the transport of active pharmaceutical compositions through the body surface of an individual, comprising applying ultrasound through a transdermal delivery device which is affixed to a portable, programmable ultrasonic regulator device, which itself is worn by the individual wherein said ultrasound is applied at an intensity and for a time period effective to enable movement of a therapeutic quantity of said active pharmaceutical composition from the transdermal delivery device through the skin, for the purpose of effecting regulated, and timed drug delivery to the individual.

2. The method of claim 1, wherein said ultrasound has a frequency in the range of about 20 kHz to 10 MHz.

3. The method of claim 1, wherein said intensity of said ultrasound is in the range of about 0.01 W/cm.sup.2 to 5.0 W/cm.sup.2.

4. The method of claim 1, wherein the wearable, portable sonic device is affixed onto or connects to a transdermal patch which provides the transdermal delivery of drugs to the individual.

5. The method of claim 1 wherein rechargeable batteries are used as the primary battery supply.

6. The method of claim 1, wherein the wearable, portable sonic device is controllable through programmable settings such as to the quantity of drug released by the device, the time interval of active ultrasonic drug delivery. the time interval between ultrasonic drug delivery, the frequency and intensity of the ultrasonic signal, the basal delivery schedule of drug dosing and the bolus delivery schedule of booster doses of a particular drug, with both automatic functions and a manual operation capability.

7. The method of claim 1, wherein the ultrasound is applied continuously.

8. The method of claim 1, wherein the ultrasound is pulsed.

9-51. (canceled)
